(19) United States
(12) Patent Application Publication
(10) Pub. No.: US 2024/0377384 A1
(43) Pub. Date: Nov. 14, 2024

(54) BLOOD-MEASURING SYSTEM

(71) Applicant: HEMETRIX, INC., San Diego, CA (US)

(72) Inventors: Matthew BANET, San Diego, CA (US); Ali NSAIR, Westchester, CA (US)

(73) Assignee: HEMETRIX, INC., San Diego, CA (US)

(21) Appl. No.: 18/658,922

(22) Filed: May 8, 2024

Related U.S. Application Data
(60) Provisional application No. 63/500,893, filed on May 8, 2023.

(51) Int. Cl. G01N 33/49; A61B 5/15; A61M 25/00

(52) U.S. Cl. CPC G01N 33/4905; A61B 5/15003; A61B 5/150099; A61B 5/150755; A61B 5/150992; A61M 25/0043

(57) ABSTRACT

The invention provides a system for measuring an Activated Clotting Time (ACT) value from a sample of blood. The system features a syringe component that encloses the sample of blood. Disposed within the syringe component are set of electrodes (composed, e.g., of metal pads or rings) that contact the sample of blood. An electronics module connected to the syringe component features an electrical impedance system that electrically connects to the set of electrodes and measures an impedance value indicating an electrical impedance of the sample of blood. A processing system receives the impedance value from the electronics module and: i) process it with a first algorithm to determine a viscosity value of the sample of blood; and ii) process the viscosity value with a second algorithm to determine the ACT value.

### CROSS-REFERENCE TO RELATED APPLICATIONS

The present invention claims the benefit of priority to U.S. Provisional Application Ser. No. 63/500,893, filed on May 8, 2023, which is incorporated herein by reference in its entirety.

### BACKGROUND AND FIELD OF THE INVENTION

### 1. Field of the Invention

The invention relates to the fields of aspirating blood from patients and then measuring activated clotting time (herein “ACT”), hematocrit and hemoglobin (herein “H&H”), and other biomarkers during surgical procedures and post-surgery recovery.

### 2. General Background

Cardiovascular disease is the one of largest causes of morbidity and mortality worldwide. During the last few decades, this trend has driven an evolution and rapid growth of: 1) percutaneous coronary interventions (herein “PCI”) procedures that feature coronary stenting for clearing arteries that are blocked due to atherosclerotic disease; and 2) minimally invasive procedures that feature percutaneous trans-catheter aortic valve replacements (herein “TAVR”) for aortic valve disease and leaking mitral valves.

Both PCI and TAVR procedures are typically done in a hospital's catheterization lab (herein “Cath Lab”) under the cover of heparin, a ubiquitous anticoagulant that prevents formation of blood clots in both the patient and catheters used for these procedures. Heparin is carefully dosed, usually based on the patient's weight, to maintain the patient within a narrow therapeutic anticoagulation range designed to avoid both blood clot formation and severe, potentially fatal bleeding. However, heparin is a heterogeneous molecule that has varying degrees of activity and affects each patient differently. This means coagulation within the patient must be monitored frequently to balance the risks of clot formation and excessive blood thinning that can cause catastrophic bleeding. In the Cath Lab, coagulation is typically characterized by measuring the patient's ACT, which represents the time blood takes to clot. ACT is currently measured during procedures with a standard point of care (herein “POC”) device. Typically, ACT is between 150-350 seconds. It is typically measured throughout PCI and TAVR procedures (e.g. every 15 minutes or so, typically resulting in 1-15 measurements/procedure). It is almost always measured immediately before and after the procedure's stent and balloon catheter are deployed.

During a typical PCI or TAVR procedure, a nurse initiates an intravenous (herein “IV”) catheter into a vein in the patient's arm. Medications (e.g. sedatives) and heparin are delivered to the patient through the IV. Clinicians then clean an area and administer a local anesthetic where the procedure takes place, typically in an artery in the patient's wrist or groin. An incision is then made into the skin over the artery, and the cardiologist inserts a sheath through the incision site and into the artery. Once the sheath is in the vessel, the cardiologist guides a catheter into it through the sheath. Using a live X-ray, the cardiologist threads the catheter through the artery into the heart. There, the cardiologist injects a contrast dye to determine how well the heart is functioning. Through this process, called angiography, the cardiologist visually identifies blood vessels in the heart, along with any blockages or narrowing. The procedure typically lasts 30-60 minutes.

The PCI procedure typically follows angiography, during which the cardiologist inserts into the artery a catheter featuring a balloon mounted to its tip and a stent attached to the balloon. Using images taken during angiography, the cardiologist positions the stent in the coronary artery featuring blockage. At the location of the blockage, the cardiologist inflates the balloon, which widens the artery, stretching it so blood flow can return to normal. Sometimes a cardiologist inserts the stent at the site to keep the artery from narrowing in the future. After the artery is widened, the cardiologist deflates the balloon and removes the catheter. ACT is measured throughout this procedure.

Coagulation is also monitored from patients recovering from surgeries and receiving heparin drips. Here, clinicians measure a parameter called partial thromboplastin time (herein “PTT”) between 1-5 times/day during the patient's entire hospital stay, which averages 5 days. PTT has a normal range between 10-15 seconds, and is typically considered to be a better indicator of a patient's clotting factors compared to ACT; it measured in the hospital's hematology lab. PTT is not used in the Cath Lab, mostly because of convenience and the time required for the assay that measures it.

Current POC devices for measuring ACT and H&H are remote from the patient and require a clinician to: 1) stop the procedure and aspirate a blood sample (typically 3-5 ccs) with a syringe to confirm the patient is adequately anticoagulated before commencing the procedure; 2) disconnect the syringe from the patient and manually transport it to the POC device; and 3) measure the sample with the device over a time period of 5-7 minutes. Each of these steps are associated with multiple potential complications and adverse events, such as delays, introduction of air bubbles during aspiration, and formation of blood clots that may travel to the heart (and possibly induce cardiac arrest) or brain (stroke). Often the surgeon, focused on the highly technical procedure at hand, forgets to initiate an ACT measurement. Furthermore, if a patient is under-dosed on heparin, the physician may need to increase this dose, wait five minutes for a follow-on test, and then repeat the measurement. And finally, the blood sample used to test for ACT and H&H is withdrawn from the same catheter used to deliver the stent and other equipment. This has the potential to introduce in-procedure complications, such as air entrapment. In short, the current standard of care for ACT measurements is fraught with many problematic issues, a situation that means it is common for cardiac surgeons to forego testing in critical clinical situations.

Additionally, most POC devices for measuring ACT do not simultaneously measure other blood-based parameters, such as H&H; this typically requires a completely separate POC device. Simultaneous measurement of H&H and ACT offers a very significant advantage for monitoring blood levels and any other signs of bleeding in long, complex procedures, such as open-heart surgeries. Not having to withdraw 3-5 cc blood every 5 to 15 minutes will save the patient unnecessary blood loss; this is particularly beneficial for anemic patients and, perhaps even more importantly, during future use in pediatric patients.

POC devices for measuring ACT include Abbott's I-STAT, Medtronic's ACT Plus, and Hemachron's Signature Elite, each of which is purchased by hospitals and reimbursed by private and non-private payers. Currently, none of these systems make continuous, automated measurements of ACT and H&H. Each requires discrete blood samples that a clinician measures manually and multiple times during a procedure; a disposable test cartridge (typically costing $5-10) is used and disposed of after each test.

Each year, there are about 1 million PCI procedures and 113,000 TAVRs performed in the United States. The PCI procedures, with an average published cost of $118,000/procedure, represent an estimated market of $15 billion. 13% of these procedures feature bleeding-related complications, which prolongs the procedure, incurs additional costs (adding roughly $30,000/procedure), and increases the risk of morbidity and mortality. Every PCI and TAVR procedure is conducted under the cover of heparin with patients featuring similarly narrow therapeutic ACT ranges.

Beyond minimally invasive cardiac interventions, ACTs are also measured in operating rooms for open heart surgeries during cardiopulmonary bypass (32.4% of reporting sites), cardiac catheterization (32.3%), intensive or coronary care unit (13.8%), vascular surgery or catheterization (10.1%), hemodialysis (1.1%), and other procedures (10.2%). Methodologies for measuring ACTs—especially those that cure the well-defined deficiencies of these measurements, as described below—can thus have wide-ranging case use.

Heparin and subsequent ACT measurements are also used during neurology procedures, which include thrombolytic therapy, embolization of intracranial and head/neck aneurysms, cerebral angiography, and carotid artery angioplasty/stenting. In 2021, approximately 310,000 of these procedures were performed domestically.

In 2021, there were approximately 2M post-surgical patients receiving heparin drips and undergoing PTT measurements every few hours during their hospital stay.

The market for coagulation analyzers market alone (which includes ACT-measuring devices) was $4.84 Billion in 2021, up from $2.98 Billion in 2016, at a CAGR of 10.2% from 2016 to 2021. Factors driving growth of this market are an aging population, increasing prevalence of autoimmune diseases, and increasing volume of procedures requiring hematologic monitoring.

### SUMMARY OF THE INVENTION

Based on the above, it would be beneficial to have a ‘Smart Syringe Measurement Device’ (herein “SSMD”) that automatically aspirates blood from a patient and measures important parameters therefrom, e.g. ACT, PTT, and H&H. Candidates for the SSMD are patients undergoing cardiac and neurological procedures, and those recovering post-surgery.

The SSMD's measurement system features a collection of sensors disposed within a compact electronics module. This includes an innovative marriage of: 1) optical, impedance, and mechanical sensors; 2) machine-learning algorithms; and 3) automation. Microfluidics/electrochemical cells and aptamer-based assays within the SSMD may yield additional measurements of PTT, glucose, cortisol, brain natriuretic peptide (herein “BNP”), blood pH, lactate/lactic acid, and potassium/chlorine/sodium ions. Automated, multiplexed measurements of these parameters yield real-time, rapid measurements of anticoagulation levels in a closed-loop system to address and treat patients on heparin. With the SSMD, the surgeon can receive real-time, critical information and focus on the highly technical procedure at hand, with fewer interruptions, improved procedural turn-around time that minimizes human error. In embodiments, the surgeon programs a ‘profile’ of measurements into the SSMD with a touchpanel display; the system then automatically makes these measurements and displays results without any further human interaction. This approach features additional benefits of reducing volumes of manual blood aspirations and thus the risk of potentially catastrophic air or blood clots entering the patient. Additionally, the SSMD can be integrated into a closed-loop system that provides real-time measurements of critical biomarkers and rapid response to derangements to maximize efficiency and remove human error and delays.

In summary, the SSMD offers the following value propositions: (1) reducing complication risks, time and resource utilization, and costs of PCIs, TAVRs, and other interventional procedures through continuous, on-patient monitoring of ACT and H&H; (2) reducing complications of interventions related to arterial air bubbles and embolic clotting, a potentially lethal side effect that can cause a heart attack, stroke, or death; (3) improving resource utilization and automated completion of tests for ACT and H&H in critical clinical situations; and (4) ultimately demonstrating a positive effect on patient outcomes.

More specifically, in one aspect, the invention provides a system for automatically measuring a value of ACT or another property from a patient's blood sample that features a blood-extraction component controlled by a processing system that extracts the blood sample from the patient. A measurement component, also controlled by the processing system, connects to the blood-extraction component, receives the blood sample from the blood-extraction component, and deposits it in a measurement cell. It features sensors that measure signals related to coagulation from the blood sample with in the measurement cell. Computer code running on the processing system analyzes the signals to determine ACT from the blood sample.

In embodiments, the blood-extraction component includes a syringe coupled to a motor-controlled actuator and motorized valve. Here, the processing component controls the motor-controlled actuator to remove the blood sample from the patient. The processing component also controls the motor-controlled actuator and the motorized valve to pass blood to the measurement component after it is removed from the patient.

In other embodiments, the blood-extraction component includes a catheter, a manifold that connects to the catheter, and a syringe coupled to both the manifold and a motor-controlled actuator. During a measurement, the catheter inserts into one of the patient's arteries or veins to extract the blood sample. The blood-extraction component can also include a retractable sheath that, during a measurement, inserts into the catheter. The retractable sheath extracts blood once inserted into the catheter, and is then withdrawn from the catheter once blood is extracted.

In embodiments, sensors within measurement component for measuring the blood sample within the measurement cell that determine signals related to coagulation include the following: 1) an optical sensor; 2) an electrical sensor for measuring impedance; 3) an electrical sensor for measuring reactance; 4) a camera for capturing images; and 5) a mechanical sensor for measuring propagation of an object within the measurement cell. Here, computer code running on the processing system operates an algorithm that analyzes the signals related to coagulation to calculate ACT. The sample cell can also enclose a compound for activating clotting within the blood sample once it is deposited in the sample cell. The compound, for example, can be kaolin (i.e. clay particles), silica, celite, diatomaceous earth, ellagic acid, clay, and thrombin. Alternatively, the sample cell encloses a compound configured to chemically react with the blood sample once it is deposited in the sample cell, e.g. a chemical reagent such as an aptameric compound, or a chemical derivative thereof.

In another aspect, the invention provides a system for automatically measuring a parameter related to coagulation from a blood sample. The system features a computer-controlled blood-extraction component that extracts the blood sample from a patient and a computer-controlled measurement component that receives the blood sample from the blood-extraction component. A sensor within the system measures signals from the blood sample that relate to coagulation to determine a value of the parameter. In embodiments, the system is worn entirely on the patient's body.

In another aspect, the invention provides a related system for automatically measuring ACT and any other blood-based component from a patient's blood samples. The system includes similar components like those described above, along with a cassette system featuring a plurality of measurement cells, each configured to sequentially receive a blood sample from the blood-extraction component. A measurement component controlled by the processing system is coupled to the cassette system and includes sensing components like those described above to measure ACT values from blood samples within different measurement cells within the cassette.

In embodiments, the cassette system further includes a motorized component and computer code configured to: i) position a first measurement cell proximal to the sensing component; ii) automatically move the cassette system; and then iii) position a second measurement cell proximal to the sensing component. This process the repeats for each measurement cell within the cassette.

In particular embodiments, the cassette system is configured as a circular array of measurement cells. Here, it features a motor controlled by the processing system that rotates the circular array of measurement cells between measurements. Alternatively, the cassette system is a linear array of measurement cells, and includes a motor controlled by the processing system that translates the linear array of measurement cells between measurements.

In related embodiments, the blood-extracting component features a first pipetting system for depositing a blood sample into a measurement cell, and a second pipetting system for depositing a chemical reagent (such as aptamer) into a measurement cell. Systems like the standard pipetting systems that extract and deposit liquids can be used for this application. In embodiments, for example, the blood-extraction component includes a syringe coupled to a motor-controlled actuator and a motorized valve; the processing component includes computer code that controls the motor-controlled actuator to remove the blood sample from the patient. As with the previous aspects of the invention, the blood-extraction component can include a manifold that connects to a catheter and syringe to draw blood from the patient. Typically, in this case, the catheter inserts into an artery or vein of the patient to extract the blood sample, and may include a retractable sheath that inserts insert into the catheter (and in embodiments, past one or more valves in the vein so that it samples flowing blood), extracts blood, and is withdrawn from the catheter once blood is extracted.

In embodiments, the sensors used with the measurement component are similar to those described above to determine signals related to coagulation, and include: 1) an optical sensor; 2) an electrical sensor for measuring impedance; 3) an electrical sensor for measuring reactance; 4) a camera for capturing images; and/or 5) a mechanical sensor for measuring propagation of an object (e.g. a magnetically controlled ball or rod) within the measurement cell.

In another aspect, the invention provides a system for automatically measuring parameters related to coagulation from at least two blood samples that features a computer-controlled blood-extraction component that sequentially extracts multiple blood samples from a patient, a cassette system that sequentially loads the blood samples into separate measurement cells, and a computer-controlled measurement component that sequentially measures the blood samples from the separate measurement cells to determine values of the parameter. In embodiments, the system is worn entirely on the patient's body.

In another aspect, the invention provides a specific measurement cell for measuring blood-based components. The measurement cell features at least two optically transparent windows that enclose the blood sample and at least two electrically conductive electrodes that contact the blood sample. An optical measurement system coupled to the measurement cell features a light source that emits radiation at two (or more) distinct optical frequencies and is oriented so that the radiation passes through a first transparent window, through the blood sample, through a second transparent window, and onto a photodetector that measures a first property of the blood sample. An imaging system, also coupled to the measurement cell, includes a camera that collects an image of the blood sample and, in response, measures a second property of the blood sample. An impedance/reactance measurement system in electrical contact with the conductive electrodes senses a first impedance/reactance signal from a first electrode, and a second impedance/reactance signal from a second electrode. It features an impedance/reactance circuit that receives and processes the first and second impedance/reactance signals to measure a third property of the blood sample. A processing system in electrical contact with the optical measurement system, the imaging system, and the impedance/reactance measurement system receives numerical values indicating the first, second, and third properties of the blood sample. In embodiments, the processing system operates computer code that collectively analyzes the first, second, and third properties of the blood sample to determine a coagulation parameter and at least one other parameter, e.g. ACT, PTT, hemoglobin (by itself), hematocrit (by itself), H&H, or another property of the blood sample.

In embodiments, the optical measurement system is configured to measure an optical absorption spectrum representing the first property of the blood sample. For example, the optical absorption spectrum can indicate how the blood sample absorbs radiation near λ=400 nm, λ=500 nm, and λ=600 nm. The imaging system typically collects multiple images of the blood sample over time, e.g. with a sampling rate of at least 0.5 Hz. Here, imaging system can include an image-processing software system operating computer code that collectively analyzes the multiple images and, in response, generates a time-dependent trace representing the second property of the blood sample. Alternatively, the imaging system can measure a color or optical intensity representing the second property of the blood sample.

In related embodiments, the first electrode injects an electrical current into the blood sample, and the second electrode measures electrical signals that depends on the injected electrical current. Here, the impedance/reactance measurement system processes the electrical signals to measure an electrical impedance or reactance representing the third property of the blood sample, which can be its capacitance, electrical resistance, or resonant frequency. In still related embodiments, the first electrode sequentially injects electrical currents having different oscillation frequencies into the blood sample, and the second electrode measures a separate electrical signal corresponding to each oscillation frequency. Using this information, the impedance/reactance measurement system can measure an impedance or reactance spectrum representing the third property of the blood sample.

In other aspects, the invention provides a similar system, only coupled to a blood-extraction component featuring a computer-controlled suction device (e.g. syringe) that automatically extracts a blood sample from the patient.

In a related aspect, the invention provides a system for measuring a blood sample from a patient that includes: 1) a measurement cell coupled to each of an optical measurement system, an imaging system, and an impedance/reactance measurement system; and 2) a processing system in electrical contact with each of the optical measurement system, the imaging system, and the impedance/reactance measurement system, and configured to receive numerical values from each system and collectively process them to determine a coagulation property of the blood sample. This system, for example, can be worn entirely on the patient's body.

In another aspect, the invention provides a system that simultaneously measures a coagulation parameter (e.g. ACT, PTT) and hemoglobin from a patient's blood sample. As with system described above, this aspect features a measurement cell with a first optically transparent window positioned on one side of the blood sample, and a second optically transparent window positioned on an opposing side of the blood sample. A light source positioned on one side of the measurement cell emits broadband optical radiation that passes through the first and second optically transparent windows and the blood sample. An optical detector positioned on a second side of the measurement cell detects the optical radiation after it passes through the first and second optically transparent windows and the blood sample, and selectively detects a set of optical wavelengths featuring a first band of optical wavelengths that yields a first signal, and, at a later time, a second band of optical wavelengths that yields a second signal. A processing component electrically integrated with the optical detector operates computer code that: i) receives the first and second signals; ii) processes them (or values calculated therefrom) to generate an absorption spectrum of the blood sample; iii) analyzes the absorption spectrum to determine a hemoglobin parameter; iv) processes values of one of the first and second signals measured at different times (or values calculated therefrom) to generate a time-dependent waveform; and v) analyzes the time-dependent waveform to determine the coagulation parameter.

In embodiments, the optical detector includes a photodiode partially covered by a computer-controlled optical filter. Here, for example, the computer controls the optical filter to pass the first band of optical wavelengths, and then, at a later time, controls the optical filter to pass the second band of optical wavelengths. In total, this system can selectively detect at least 10 bands of optical wavelengths, ranging from λ=400-700 nm. Alternatively, the optical detector features an optical component (e.g. a diffraction grating or prism) that spatially disperses the broadband optical radiation depending on its optical wavelength, and a photodetector (e.g. a charge-coupled device (herein a “CCD camera”) or one or more sensitive optical detectors, e.g. photomultiplier tubes an avalanche photodiodes) that selectively detect portions of the dispersed broadband optical radiation. In embodiments, the processing component identifies a peak between λ=540-580 nm and determines its amplitude to yield the hemoglobin parameter. Here, for example, the processing component can analyze when the time-dependent waveform or a mathematical derivative thereof featuring an amplitude that is less than a pre-determined parameter, or undergoes a rapid change that exceeds a pre-determined parameter, to determine the coagulation parameter.

In a related aspect, the invention provides a system for measuring both a coagulation parameter and hemoglobin parameter from a patient's blood sample. The system features a computer-controlled blood-extraction component that extracts the blood sample from the patient and deposits it into a measurement cell, a light source emitting broadband optical radiation that passes through blood sample, an optical detector that receives the broadband radiation after it passes through the blood sample and selectively detects a set of optical wavelengths featuring a first band of optical wavelengths that yields a first signal, and a second band of optical wavelengths that yields a second signal. A processing component electrically integrated with the optical detector operates computer code that processes the first and second signals, or numerical values calculated therefrom, to determine the hemoglobin parameter and the coagulation parameter. As with the above-mentioned system, this system can also be worn entirely on the patient's body.

In another aspect, the invention provides a mechanical system for measuring a coagulation parameter from a blood sample from a patient. The system features a measurement cell containing the blood sample and a magnetically active material (e.g. a metal ball or rod) immersed in the blood sample. A magnet, external and proximal to the measurement cell, generates a magnetic field that moves the magnetically active material immersed in the blood sample, and an imaging system positioned collects time-dependent images of the blood sample and the moving magnetically active material therein. A processing component electrically integrates with the imaging system and includes computer code that: i) receives the time-dependent images; ii) processes the time-dependent images to extract a time-dependent signal indicating movements of the magnetically active material; and iii) analyzes the time-dependent signal to determine the coagulation parameter.

In embodiments, the system includes a linear actuator attached to the magnet and controlled by computer code operating on the processing component. Typically, both the linear actuator and attached magnet are positioned underneath the measurement cell. To move the magnet, the processing component applies a signal to the linear actuator; this drives the magnet to move back and forth underneath the measurement cell, thereby causing the magnetically active material to move within the blood sample. For example, the processing component can apply a signal to the linear actuator that causes the magnet to periodically move back and forth underneath the measurement cell at a pre-determined frequency, thereby causing the magnetically active material to move within the blood sample at a comparable frequency. Here, the imaging system collects time-dependent images of the blood sample and the moving magnetically active material therein at a sampling rate that is greater than the pre-determined frequency. In embodiments, the time-dependent signal extracted from the images indicates movements of the magnetically active material impeded by clots forming in the blood sample. In related embodiments, the analyzing step conducted by the processing component determines a decay of an amplitude of the time-dependent signal, which in turn yields the coagulation parameter. For example, the analyzing step can involve calculating a mathematical Fourier Transform of the time-dependent signal, and then calculating a decay component from the Fourier Transform. The decay component or a processed version thereof is an estimate of the coagulation parameter. In other embodiments, the analyzing step involves ‘fitting’ the time-dependent signal to a mathematical equation to estimate a decay component. Here, fitting means using an algorithm (embodied by computer code running on the processing system) to iteratively vary the parameters of a mathematical function that describes the physical event at hand, i.e. clotting blood. The algorithm varies the parameters until an error (typically called ‘χ²’) representing the difference between the data measured by the system and the mathematical function is minimized. In this case, the decay component determined from the fitting, or a parameter calculated therefrom, is an estimate the coagulation parameter.

In other embodiments, the magnet is a stationary electromagnet, and the magnetic field is varied when a voltage is applied to the electromagnet. For example, the processing component can apply a time-dependent voltage to the electromagnet that causes the magnetic field it generates to vary according to a pre-determined frequency, thereby causing the magnetically active material to move within the blood sample at a comparable frequency. The imaging system then collects time-dependent images of the blood sample and the moving magnetically active material at a sampling rate that is greater than the pre-determined frequency. The time-dependent signal indicates movements of the magnetically active material impeded by clots forming in the blood sample, and the analyzing step determines a decay of an amplitude of the time-dependent signal to determine the coagulation parameter. As before, the analyzing step can calculate a mathematical Fourier Transform of this time-dependent signal, and then extract a decay component from the Fourier Transform. Further processing of the decay component yields the coagulation parameter. Or the analyzing step can include fitting the time-dependent signal to a mathematical equation to estimate a decay component, and then using this value to estimate the coagulation parameter.

In embodiments, the measurement cell further includes a compound that activates clotting of the blood sample, e.g. kaolin, silica, celite, diatomaceous earth, ellagic acid, clay, thrombin, an aptameric compound, or a chemical derivative thereof.

In a related aspect, the invention provides a system for measuring a coagulation parameter from a blood sample from a patient that features: 1) a blood-extraction component that automatically extracts the blood sample from the patient; 2) a measurement cell featuring a magnetically active material that receives the blood sample; 2) a magnet that generates a magnetic field that moves the magnetically active material within in the blood sample; 4) an imaging system positioned to collect time-dependent images of the blood sample and the moving magnetically active material therein; and 5) a processing component configured to receive the time-dependent images and process them to determine the coagulation parameter. As before, the system can be worn entirely on the patient's body.

In another aspect, the invention provides an impedance/reactance system for measuring a coagulation parameter (e.g. ACT, PTT) from a blood sample from a patient. The system features a removable measurement cell containing the blood sample and featuring a first ‘drive’ electrode that injects a first electrical current into the blood sample, and a first ‘sense’ electrode that senses a first electric signal from the blood sample. During a measurement, a mounting component receives the removable measurement cell. The mounting component features a first ‘drive’ metal contact that makes electrical contact with the first drive electrode, and a first ‘sense’ metal contact that makes electrical contact with the first sense electrode when the mounting component receives the removable measurement cell. The contacts, for example, can be retractable or spring-loaded pogo pins. An impedance/reactance circuit in electrical contact with one or more drive metal contacts controls the first electrical current injected into the blood sample, and also processes the first electrical signal sensed from the blood sample. A processing component in electrical contact with the impedance/reactance circuit operates computer code that: i) receives the electrical signal or a signal derived therefrom; ii) processes the electrical signal or a signal derived therefrom to generate a time-dependent signal; and iii) analyzes a property of the time-dependent signal to determine the coagulation parameter.

In embodiments, the removable measurement cell is of a cuboid geometry (e.g. a three-dimensional rectangle or similar shape), with a first side of the cuboid including the first sense electrode and the first drive electrode. The second side of the cuboid includes a second drive electrode that injects a second electrical current into the blood sample and a second sense electrode that senses a second electric signal from the blood sample. Here, the mounting component includes a second drive metal contact (similar to that described above) that makes electrical contact with the second drive electrode and a second sense metal contact that makes electrical contact with the second sense electrode when the mounting component receives the removable measurement cell.

In embodiments, the impedance/reactance circuit features a differential amplifier that receives the first and second electric signals, and amplifies the difference between them to generate an amplified signal. The processing component then determines an amplitude change from the first and second electric signals, e.g. a time-dependent amplitude change. It then processes the time-dependent amplitude change to determine a blood-clotting time that represents the coagulation parameter. Alternatively, the processing component determines a phase change from the first and second electric signals, e.g. a time-dependent phase change. It then processes the time-dependent phase change to determine a blood clotting time that represents the coagulation parameter.

In embodiments, the impedance/reactance circuit controls a frequency of the first electrical current. For example, it can inject electrical current at a single frequency into the blood sample. Alternatively, it can inject electrical current at a collection of frequencies (e.g. ‘sweep’ the frequencies from 5-1000 MHz) into the blood sample.

In other embodiments, the cuboid features an optically transparent window, and the sense and/or drive electrodes are an electrically conductive film disposed on the optically transparent window. For example, the electrically conductive film can be a metal film, an indium tin oxide (herein “ITO”) film, or a chemical derivative thereof.

In other embodiments, during a measurement, the computer-controlled automation component inserts the removable measurement cell into the mounting component, and then removes the removable measurement cell from the mounting component after the processing component determines the coagulation parameter.

In other aspects, the impedance/reactance system also includes a blood-extraction component featuring a computer-controlled device that automatically extracts the blood sample from the patient and then deposits it in a measurement cell.

In another aspect, the invention provides a system for measuring a coagulation parameter from a blood sample featuring a blood-extraction component that automatically extracts the blood sample from a patient and deposits it in a measurement cell. The cell features a drive electrode that injects a first electrical current into the blood sample and a sense electrode that senses an electric signal from the blood sample, an impedance/reactance circuit that injects electrical current injected into the blood sample through the drive electrode and senses the electrical signal from the blood sample through the sense electrode, and a processing component operating computer code that receives the electrical signal, processes it, and then analyzes the resulting signal to determine the coagulation parameter. As before, this system can be worn entirely on the patient's body.

In another aspect, the invention provides a system for continuously measuring coagulation (e.g. ACT, PTT) and other parameters (e.g. H&H or hemoglobin and hematocrit by themselves) from a patient. Here, ‘continuously’ means a new parameter is measured at a relatively high frequency, e.g. once every 30 seconds.

In this aspect, the invention features a first measurement cell that receives a first blood sample, a second measurement cell that receives a second blood sample, and a mounting component. An automation component places the first measurement cell in the mounting component, and once a first measurement is completed, removes the first measurement cell from the mounting component and places the second measurement cell therein. A sensor is proximal to the mounting component and positioned to sense a first signal from clotted blood in the first blood sample, and a second signal from partially clotted blood in the second blood sample. A processing component electrically integrated with the sensor operates computer code configured to: i) receive the first signal; ii) process the first signal or values calculated therefrom to determine an absolute clotting time of the first blood sample; iii) receive the second signal; and iv) analyze the second signal and the absolute clotting time to calculate an estimated clotting time of the second blood sample.

In embodiments, this system features the same or similar sensors as those described above, e.g. an optical sensor featuring a light source and a photodetector, wherein the light source is positioned on a first side of the mounting component, and the photodetector is positioned on a second side of the mounting component. Here, the first signal is a first time-dependent waveform, and the process step made by the processing component involves analyzing the first time-dependent waveform to determine a temporal feature indicating that the first blood sample is clotted. The second signal is also a time-dependent waveform, and the processing component analyzes it with a mathematical algorithm to determine the estimated clotting time. For example, the mathematical algorithm can be a numerical fitting algorithm, an algorithm using machine learning, an algorithm using artificial intelligence, or an algorithm using frequency-domain analysis (e.g. Fourier Transform).

In other embodiments, the sensor is an impedance/reactance sensor that, like those systems described above, includes a drive electrode that injects electrical current into a sample, and a sense electrode that measures the sample's response to the injected electrical current. This system—wherein both the sense and drive electrodes contact the first blood sample and at a later time the second blood sample—yields a first time-dependent waveform from the first sample. The processing component operates computer code that further analyzes the first time-dependent waveform to determine a temporal feature indicating that the first blood sample is clotted. Then, at a later time, the system measures a second time-dependent waveform, and the processing component analyzes it with a mathematical algorithm to determine the estimated clotting time. As with the optical sensor, the mathematical algorithm used here can be a numerical fitting algorithm, an algorithm using machine learning, an algorithm using artificial intelligence, or an algorithm using frequency-domain analysis (e.g. a Fourier Transform).

In still other embodiments, the sensor is an imaging sensor, e.g. a camera positioned on a first side of the mounting component that collects images of the first blood sample and then the second blood sample. Here, as before, the first signal is a first time-dependent signal extracted from images collected by the camera from the first blood sample, and the process step made by the processing component involves analyzing the first time-dependent signal to determine a temporal feature indicating that the first blood sample is clotted. The second signal is a second time-dependent signal, and the processing component analyzes it with a mathematical algorithm similar to those described above to determine the estimated clotting time.

In another aspect, the invention provides a system for measuring clotting times from blood samples extracted from a patient that features a first measurement that receives a first blood sample and a second measurement cell that receives a second blood sample, a sensor positioned to sense an absolute clotting time from clotted blood the first blood sample, and at a later time a second signal from partially clotted blood from the second blood sample, and a processing component electrically integrated with the sensor and configured to analyze the second signal and the absolute clotting time to calculate an estimated clotting time of the second blood sample. As before, the system can be configured to be worn entirely on the patient's body.

In another aspect, the invention provides a system with a blood-extraction component similar to that described above, and a blood-measuring system featuring a first data interface that receives the blood sample from the blood-extraction component and process it to determine a parameter within the blood sample. The system also includes a vital signs monitor featuring a second data interface that connects to the patient to measure physiological signals. A processing component receives a first set of numerical information indicating the blood parameter through the first data interface, and a second set of numerical information indicating the physiological signals through the second data interface. It then collectively analyzes the first and second sets of numerical information to characterize the patient.

In embodiments, the blood parameter is a level of potassium, and the physiological signal is an ECG waveform. Here, the processing component operates computer code that measures a T-wave within the ECG waveform, and from this determines its amplitude. The processing component then collectively analyzes the level of potassium and the amplitude of the T-wave to determine, at a later time, an estimated level of potassium in the patient's blood. With this approach, the processing component can determine the estimated level of potassium at a relatively high rate (e.g. a new measurement every minute) compared to the rate that the blood-measuring system determines the level of potassium.

In another embodiment, the blood parameter is a level of hematocrit, and the physiological signals are an impedance/reactance waveform. Here, the processing component processes the impedance/reactance waveform to determine a thoracic impedance and its amplitude. The processing component then analyzes the level of hematocrit and the amplitude of the thoracic impedance to determine a fluid parameter of the patient, e.g. the patient's fluid level, blood volume, and fluid responsivity. Here, ‘fluid responsivity’ means how the patient responds to fluids, e.g. whether hemodynamic parameters like stroke volume, cardiac output, and blood pressure increase or decrease when fluids are administered. Like the measurement of potassium, the measurement of hematocrit is made at a high frequency (e.g. every minute). In related embodiments, the processing component processes the impedance/reactance waveform to determine a stroke volume or a cardiac output value from the patient, or a change in these parameters, and then analyzes the level of hematocrit and the change of either the stroke volume or cardiac output value to determine a fluid parameter of the patient.

In another embodiment, the blood parameter is either lactic acid and lactate, and the physiological signals are within an ECG waveform, and specifically the QRS-complex within the ECG waveform. Here, the processing component analyzes the ECG waveform and its QRS-complex to determine a heart rate or heart rate variability, and then further analyzes the level of lactic acid or lactate and the heart rate (and/or variability) to estimate a level of sepsis in the patient. In related embodiments, for the measure of sepsis, the measurement of ECG and heart rate can be replaced (or augmented) with measurements of skin temperature and core temperature (with a temperature sensor), respiration rate (taken from the ECG waveform and/or the impedance/reactance waveform), SpO₂ (taken from a waveform called a photoplethysmogram (herein “PPG”) measured with a pulse oximeter), or another physiological waveform.

In yet another embodiment, the processing component is further configured to analyze the level of hemoglobin and/or hematocrit and a physiological parameter such as heart rate, respiration rate, SpO₂, blood pressure, stroke volume, and cardiac output to determine a level of hemorrhage in the patient.

In each of these cases, one or both of first data interface and the second data interface can be a wireless interface, e.g. Bluetooth®, Wi-Fi, and cellular interface. The vital signs monitor used in this application can be a wearable system worn completely on the patient's body.

In related embodiments, the system can include a wireless transceiver (e.g. a Bluetooth® transceiver, a Wi-Fi transceiver, or a cellular transceiver) that wirelessly transmits the ACT value (or a parameter related to this) to a remote display, such as a remote computer, tablet computer, cellular telephone, or a display associated with a hospital's electronic medical records (herein “EMR”) system.

In another aspect, the invention provides a system for measuring a parameter from a blood sample from a patient. The system features a blood-extraction component similar to that described above, and a reagent-distribution component that includes, e.g. a liquid reagent. Here, the measurement cell features an optically transparent portion and can receive both the blood sample from the blood-extraction component and the reagent from the reagent-distribution component. An imaging system positioned proximal to the cell's optically transparent portion collects images of the blood sample after it is mixed with the reagent. A processing component electrically integrated with the imaging system operates computer code configured to: i) receive the images; and ii) analyze the images with an algorithm to determine the parameter.

In embodiments, the reagent comprises an aptamer, e.g. a DNA or RNA aptamer. The aptamer can be chemically bound to a colorimetric compound configured to undergo a color change when the aptamer binds to the parameter. For example, the parameter can be thrombin, and the aptamer is configured to bind to a fibrinogen binding site and is 5′-GGTTGGTGTGGTTGG-3. Or the parameter can be thrombin, and the aptamer is configured to bind to a heparin binding site and is 5′AGTCCGTGGTAGGGCAGGTTGGGGTGACT-3.

In embodiments, the imaging system is a camera, and the algorithm is an image-processing algorithm configured to detect a colorimetric change of the blood sample after it is mixed with the reagent. Or the imaging system is a light source and a photodetector, and the algorithm determines an absorption spectrum of the blood sample after it is mixed with the reagent. Or the reagent is a clotting agent, and the imaging system measures the time associated with clotting, as described above.

In a related aspect, the invention provides a similar system featuring a blood-extraction component and a measurement cell with an optically transparent portion, and additionally includes a centrifuge component that receives the measurement cell, and afterwards centrifuges it and the blood sample to separate blood cells from plasma within the measurement cell. An imaging system (e.g. a camera or combined light source and photodetector) positioned proximal to the optically transparent portion collects images of the blood sample after it is centrifuged. A processing component electrically integrated with the imaging system includes computer code configured to: i) receive the images; and ii) analyze the images with an algorithm to determine the parameter. In embodiments, the algorithm is an image-processing algorithm that detects a ratio of blood cells and plasma.

In another aspect, the invention provides a similar system to that described above, augmented to include a reagent-distribution system. The system also includes a measurement cell that receives the blood sample from the blood-extraction component and the reagent from the reagent-distribution component, and features an optically transparent portion and a magnetically active material. A magnet within the system generates a magnetic field that moves the magnetically active material within the blood sample and reagent to mix them together. An imaging system, similar to that described above, is positioned proximal to the optically transparent portion and collects images of the blood sample after it is mixed with the reagent. A processing component electrically integrated with the imaging system operates computer code is configured to: i) receive the images; and ii) analyze the images with an algorithm to determine the parameter.

In embodiments, the reagent can be an aptamer (e.g. a DNA or RNA aptamer) or a clotting agent, similar to those described above. In other embodiments, the system includes a linear actuator positioned underneath the measurement cell and attached to the magnet and controlled by the processing component. Here, the processing component applies a signal to the linear actuator to cause the magnet to move back and forth underneath the measurement cell, thereby causing its magnetic field to vary and the magnetically active material to move within the blood sample. Alternatively, the magnet is a stationary electromagnet featuring a magnetic field that varies when a voltage is applied to the magnet.

In another aspect, the invention provides a system (e.g. a closed-loop system) for delivering heparin to a patient. The system features a blood-extraction component with a computer-controlled device that automatically extracts a blood sample from the patient, and a heparin-delivery component featuring a source of heparin and a computer-controlled pump that delivers heparin to the patient. A patient-interface component including a catheter that inserts into the patient's venous system serves two purposes: it extracts the blood sample and delivers heparin. This component features three computer-controlled valves, with the first and second computer-controlled valves connected to the blood-extraction component and the third computer-controlled valve connected to the heparin-delivery component. A measurement cell receives the blood sample from the blood-extraction component, and a sensor component positioned proximal to the measurement cell measures a coagulation parameter (e.g. ACT, PTT) from the blood sample. A processing component electrically integrated with the blood-extraction component, heparin-delivery component, patient-interface component, and sensor component operates computer code configured to: i) control the patient-interface component and the blood-extraction component to extract the blood sample from the patient; ii) control the sensor component to measure the coagulation parameter from the blood sample; iii) analyze the coagulation parameter by comparing it to pre-determined information related to values of the coagulation parameter and doses of heparin; and iv) based on the analyzing, control the patient-interface component and the heparin-delivery component to deliver a specific dose of heparin to the patient.

In embodiments, the processing component opens the first computer-controlled valve and closes the second computer-controlled valve so that the blood-extraction component can extract the blood sample from the patient. As with systems described above, the blood-extraction component can include a retractable sheath that inserts into the catheter and is in electrical contact with the patient-interface component. The patient-interface component controls the retractable sheath to extract blood once it is inserted into the catheter, and then withdraws it from the catheter once blood is extracted. At this point, the processing component closes the first computer-controlled valve and opens the second computer-controlled valve to deliver the blood sample extracted from the patient to the measurement cell, where it is then measured with an optical, electrical, or mechanical technique, as described above, to determine the coagulation parameter. The system then automatically delivers heparin to the patient based on the value of the coagulation parameter.

In a related aspect, the invention provides a system for delivering heparin to a patient that features a blood-extraction component that extracts a blood sample from the patient and a heparin-delivery component with a source of heparin that delivers heparin to the patient. A patient-interface component includes a catheter that inserts into the patient's venous system to extract the blood sample and deliver heparin. At least two computer-controlled valves connect to the blood-extraction component and the heparin-delivery component. Also within the system is a measurement cell configured to receive the blood sample from the blood-extraction component, a sensor component configured to measure ACT from the blood sample, and a processing component electrically integrated with the blood-extraction component, heparin-delivery component, patient-interface component, and sensor component. The processing component operates computer code configured to: i) control the patient-interface component and the blood-extraction component to extract the blood sample from the patient; ii) control the sensor component to measure the ACT from the blood sample; iii) analyze the ACT; iv) based on the ACT, control the patient-interface component to deliver a specific dose of heparin to the patient. The system can be worn entirely on the patient's body.

In other aspects, the invention provides specific methods for performing the various steps associated with each of the systems, above. Such methods, for example, are typically performed using software systems and computer code operating on the processing system that controls the blood-extraction component, the measurement component, and any pumps and valves associated with the closed-loop system for delivering heparin to the patient.

These and other advantages of the invention should be apparent from the following detailed description, and from the claims.

### BRIEF DESCRIPTION OF THE DRAWINGS

FIG. 1 is a schematic drawing of the SSMD according to the invention for automatically measuring ACT, H&H, and other blood-based compounds during a PCI procedure;

FIG. 2A is a schematic drawing of a measurement component from the SSM/ID of FIG. 1 featuring a measurement cell with optical, electrical impedance, and mechanical sensors;

FIGS. 2B and 2C are schematic drawings of measurement systems featuring, respectively, circular and linear cassettes, each incorporating a plurality of measurement cells similar to that shown in FIG. 2A;

FIG. 3 is a side-view mechanical drawing of the measurement system within the SSMD featuring a circular cassette similar to that shown in FIG. 2B;

FIG. 4 is a schematic drawing of the SSMD mounted on an IV pole and wirelessly communicating with a remote gateway device;

FIGS. 5A-F are side-view mechanical drawings of the SSMD of FIG. 1 at various stages of blood draw and measurement cycles;

FIGS. 6A and 6B show, respectively, schematic drawings of versions of a blood-extraction component used to automatically extract venous blood during and in between measurements;

FIG. 6C is a schematic drawing of a front su face of a mechanical component shown in FIGS. 6A and 6B for moving a mechanical sheath in and out of a patient's vein;

FIGS. 7A-D are photographs of POC devices of the prior art that measure ACT and H&H;

FIG. 8A is a side-view mechanical drawing of a measurement component of the SSMD featuring optical, electrical impedance, and mechanical sensors and a measurement cell;

FIG. 8B is a photograph of the measurement component that was fabricated from the mechanical drawing in FIG. 8A;

FIG. 9A is a top-view mechanical drawing of the measurement component and sensors shown in FIG. 8A;

FIG. 9B is a photograph of the measurement component that was fabricated from the mechanical drawing in FIG. 9A;

FIG. 10A is a front-view mechanical drawing of the measurement cell of FIG. 8A featuring a magnetically active metal ball used during a mechanical measurement of ACT;

FIG. 10B is a photograph of the measurement cell that was fabricated from the mechanical drawing in FIG. 10A;

FIG. 11A is a front-view mechanical drawing of the measurement cell of FIG. 10A, surrounded by both moving magnets and stationary electromagnets for moving a conductive metal ball within the cell;

FIG. 11B is a photograph of the mechanical cell of FIG. 11A, taken during an ACT measurement, showing a metal ball within the measurement cell;

FIGS. 12A-12D are time-domain plots of simulated signals calculated from a system representing coagulating blood that is, respectively, completely unclotted, moderately clotted, significantly clotted, and completely clotted;

FIGS. 12E-12H are frequency-domain plots determined from the Fourier Transforms of the time-domain plots shown in FIGS. 12A-12D;

FIG. 13A is side-view mechanical drawing of the measurement cell of FIG. 10A featuring pairs of sense and drive electrodes that perform an electrical impedance or reactance measurement;

FIG. 13B is a schematic graph of time-dependent, out-of-phase electrical current injected into the measurement cell of FIG. 13A using opposing drive electrodes within the cell;

FIGS. 14A-14H are photographs of the measurement cell of FIG. 8A filled with clotting blood and taken at times ranging from 0-327 s after the blood is aspirated from a human patient;

FIGS. 15A and 15B are, respectively, time-dependent plots of electrical impedance measured from blood samples without and with heparin using the measurement cell of FIGS. 14A-14H;

FIG. 16 is a scatter plot showing agreement between ACT calculated from waveforms similar to those shown in FIGS. 15A and 15B (y-axis) and ACT measured with a standard POC device (x-axis) similar to that shown in FIG. 7B;

FIG. 17A is a side-view mechanical drawing of the measurement cell of FIG. 10A undergoing an optical measurement;

FIG. 1713 is a time-dependent plot showing an optical signal measured from a measurement cell similar to that shown in FIG. 17A filled with clotting blood that was aspirated from a human patient;

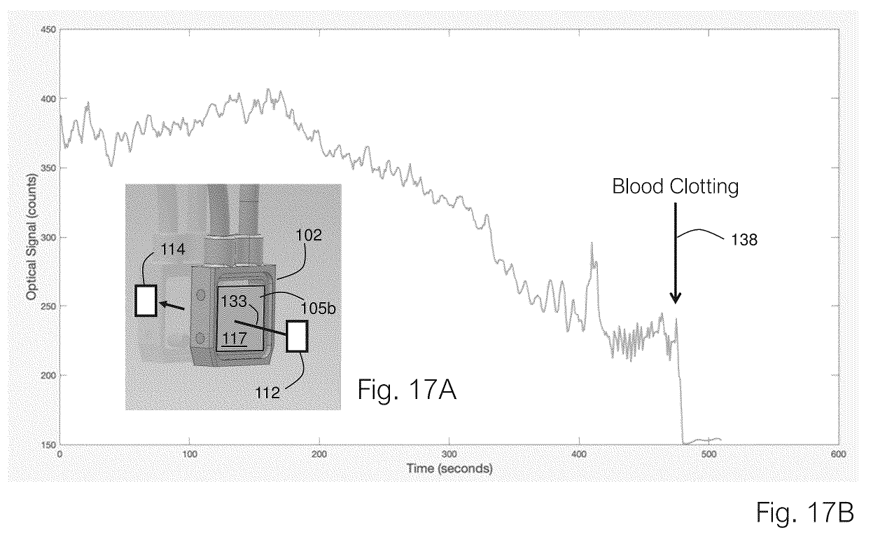

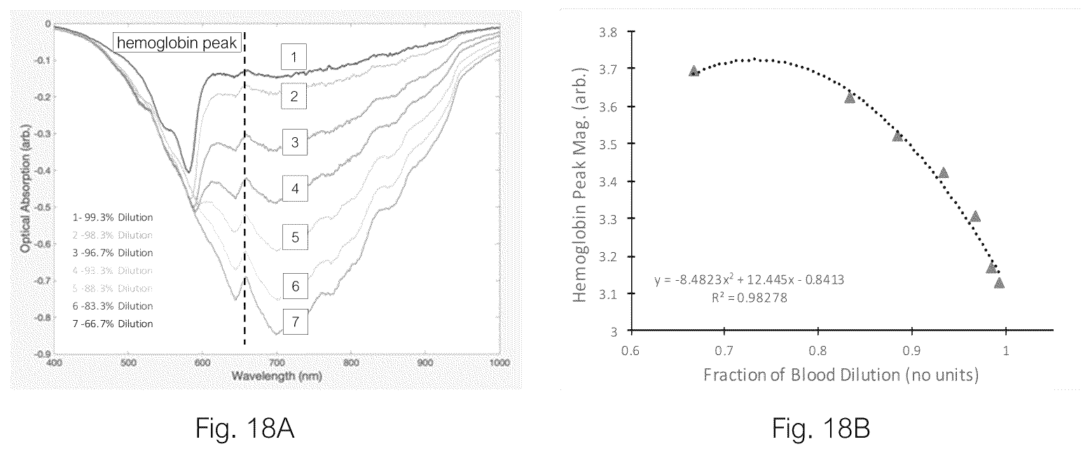

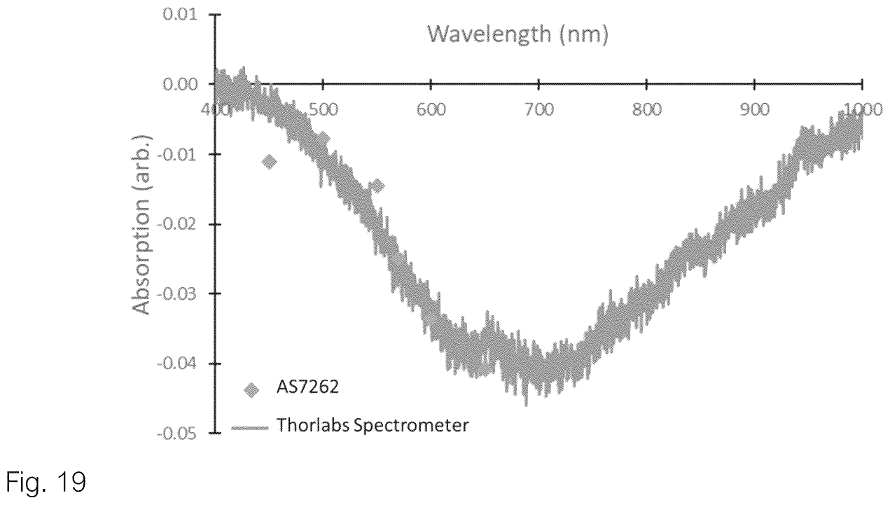

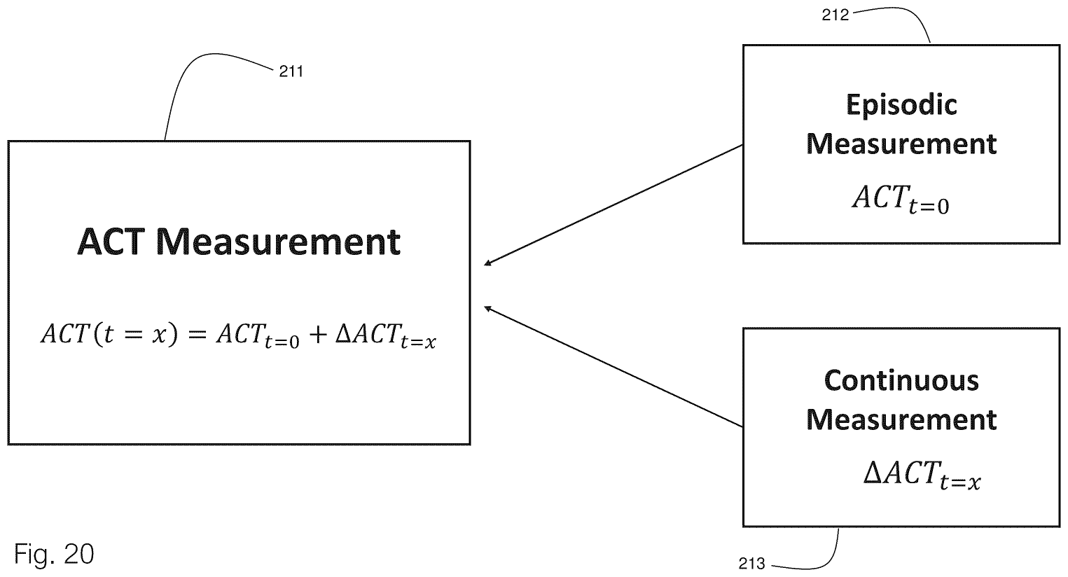

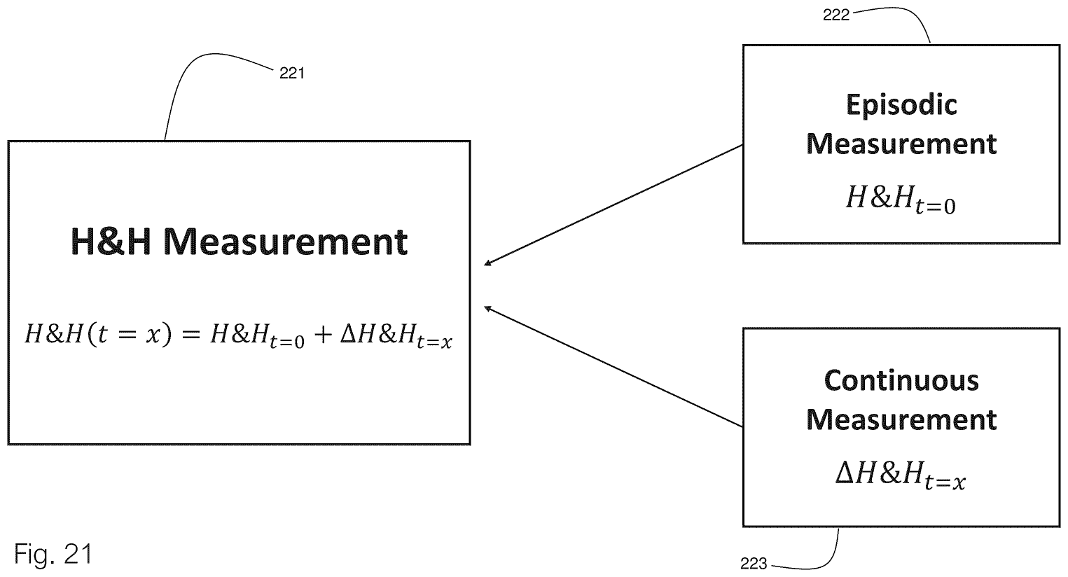

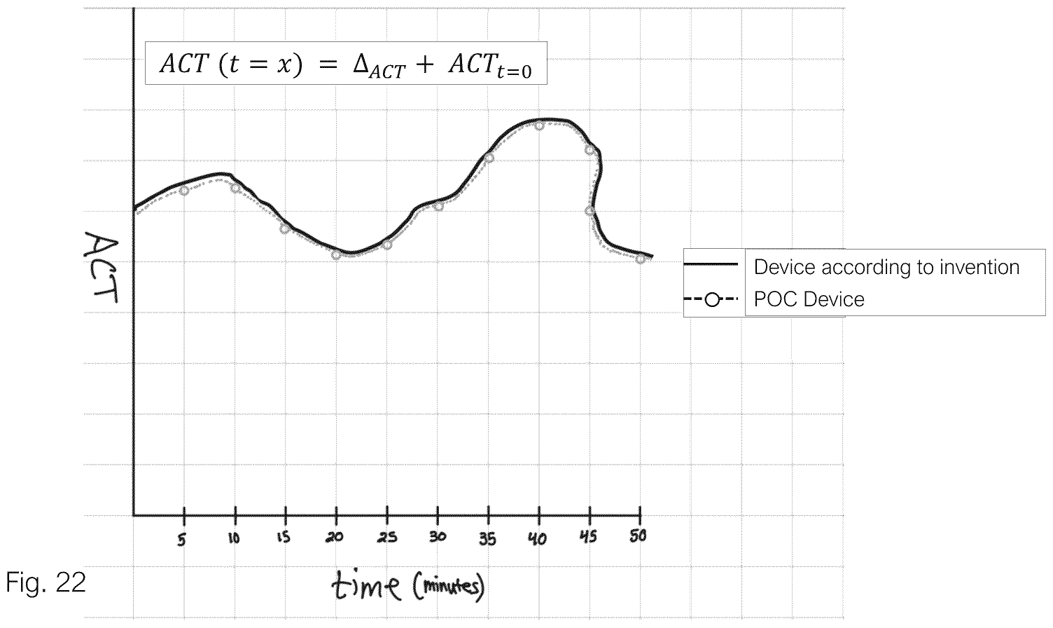

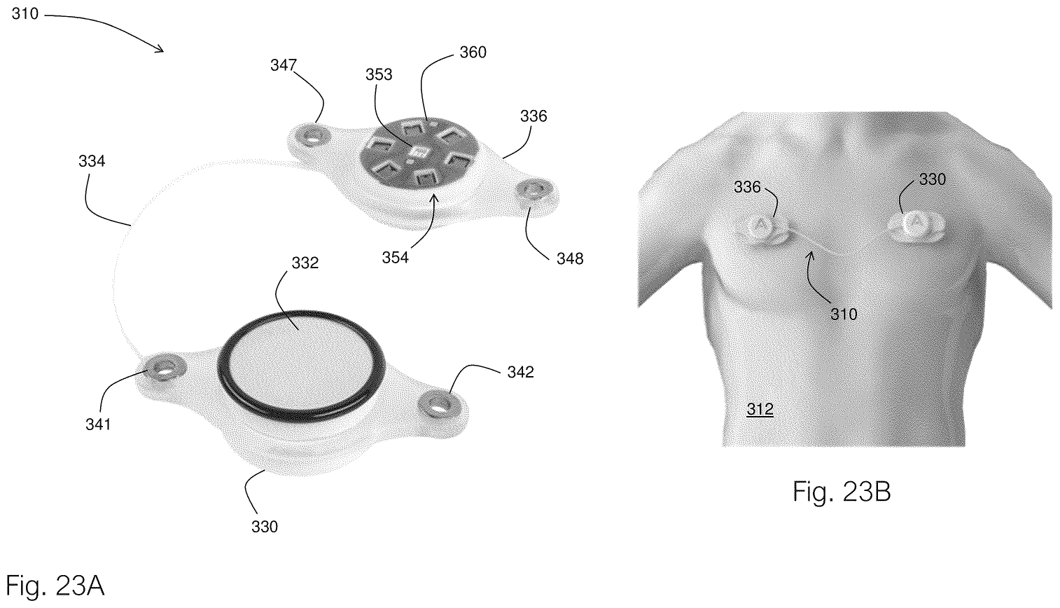

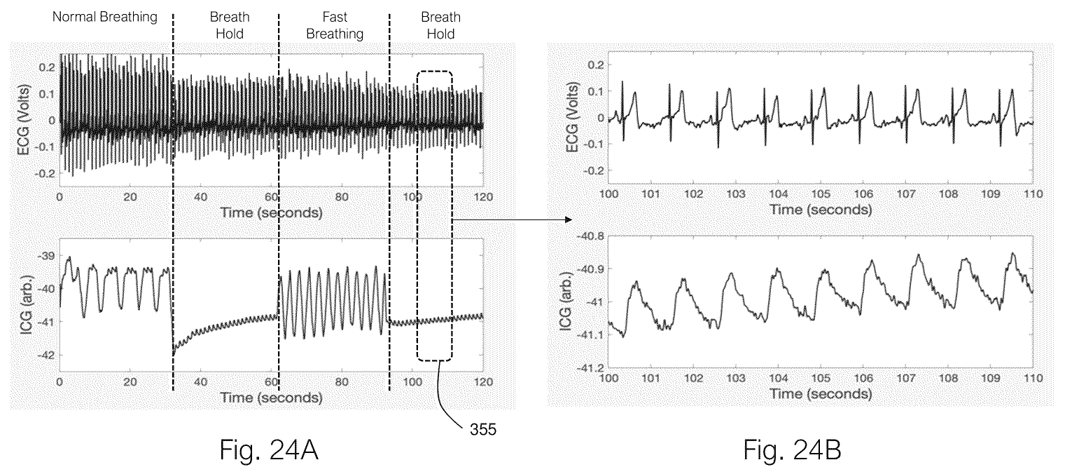

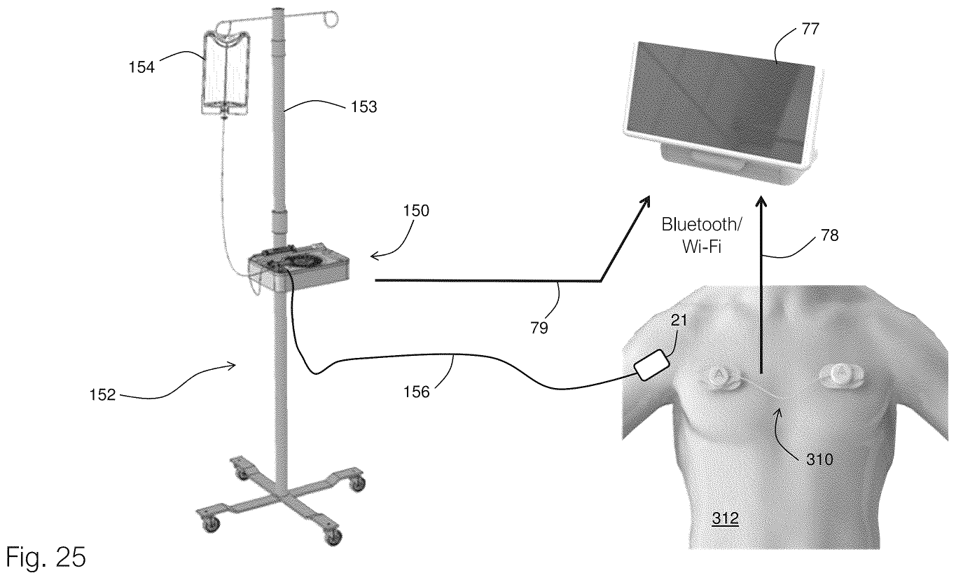

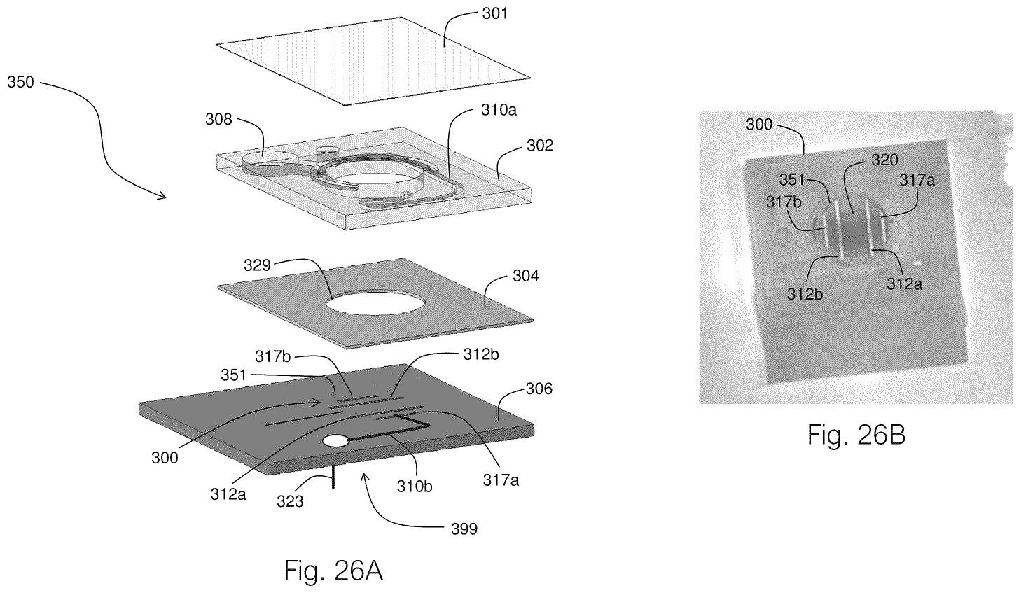

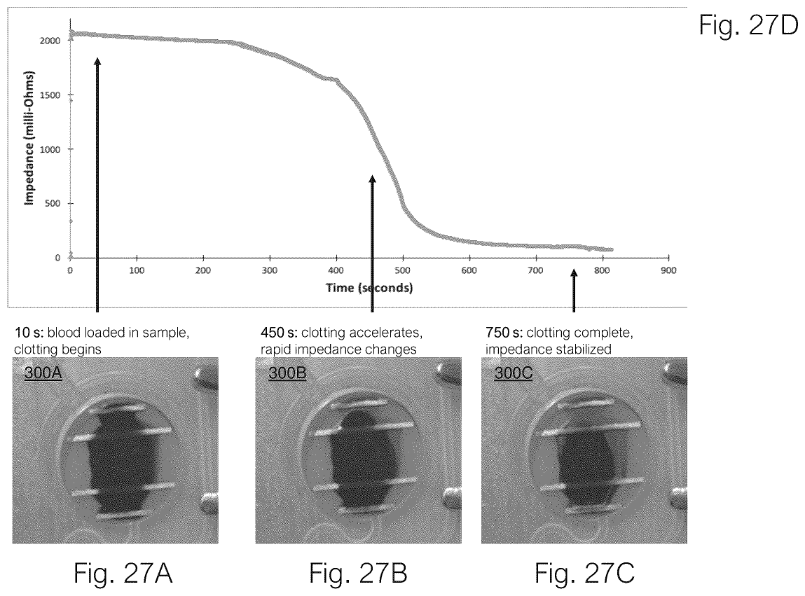

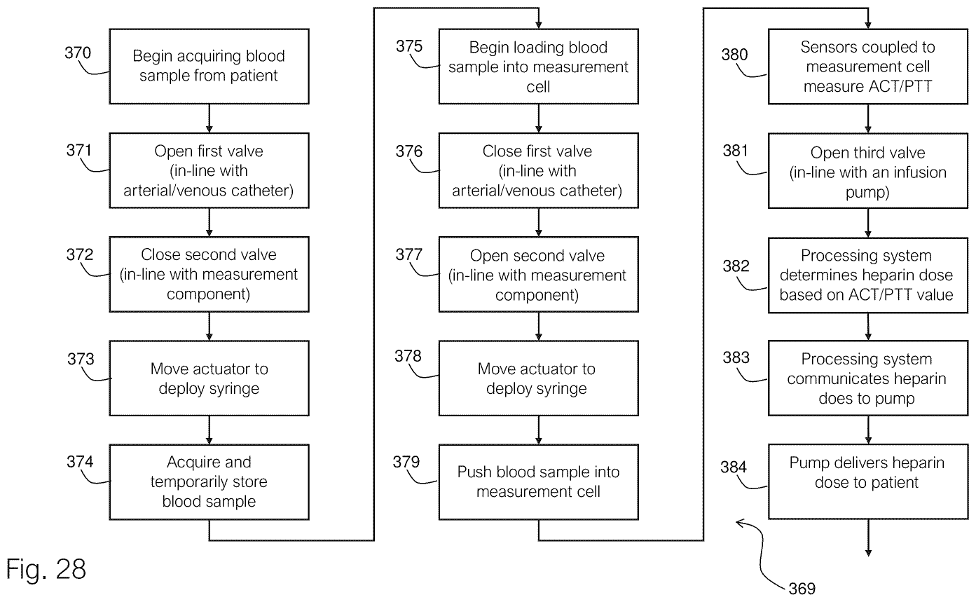

FIG. 18A is a wavelength-dependent optical absorption spectrum featuring a peak near λ=660 nm corresponding to hemoglobin measured from human blood within a measurement cell similar to that shown in FIG. 17A;

FIG. 18B is a scatter plot showing amplitudes of the hemoglobin peak indicated in FIG. 18A measured from human blood samples that were systematically diluted with deionized water;

FIG. 19 is a wavelength-dependent optical absorption spectrum measured continuously over a wavelength range of λ=400-1000 nm with a conventional absorption spectrometer (from Thorlabs, Inc.), and also measured at discrete wavelengths over a similar wavelength range with a chip-level optical detector (from AMS, Inc.);

FIG. 20 is a schematic drawing indicating an algorithm for a continuous measurement of ACT made according to the invention;

FIG. 21 is a schematic drawing indicating an algorithm for a continuous measurement of H&H made according to the invention;

FIG. 22 is a time-dependent plot showing continuous measurements of ACT made using an algorithm shown in FIG. 20, and episodic measurements of ACT made using a conventional POC device;

FIG. 23A is a mechanical drawing of wearable physiological sensor according to the invention that non-invasively measures ECG and bio-impedance waveforms, and from these calculates both vital signs and hemodynamic parameters;

FIG. 23B is a schematic drawing of the physiological sensor of FIG. 23A worn on a patient's chest;

FIG. 24A is a time-dependent plot showing ECG waveforms (top) and bio-impedance waveforms (bottom) measured with the physiological sensor of FIG. 23A from a human patient undergoing normal breathing, breath holds, and fast breathing;

FIG. 24B is a time-dependent plot showing close-tip views of the ECG (top) and bio-impedance (bottom) waveforms shown in FIG. 24A;

FIG. 25 is a schematic drawing showing the SSMD and the physiological sensor of FIG. 23A connected to a patient and wirelessly transmitting information to a remote gateway;

FIG. 26A is an exploded mechanical drawing of a wearable measurement cell according to an alternate embodiment of the invention that uses microfluidics to load blood samples into a measurement area;

FIG. 26B is a photograph of the wearable measurement cell of FIG. 26A featuring sense and drive electrodes for impedance/reactance measurements and containing human blood;

FIGS. 27A-27C are photographs of the measurement cell of FIG. 26A containing human blood and taken at different time periods to show increasing levels of coagulation;

FIG. 27D is a time-dependent plot of an electrical impedance waveform measured from the measurement cells of FIGS. 27A-27C; and

FIG. 28 is a flow chart showing steps used by a closed-loop system to measure ACT from a patient and, in response, deliver a dose of heparin to the patient.

### DETAILED DESCRIPTION OF THE INVENTION

### 1. Clinical Use Case

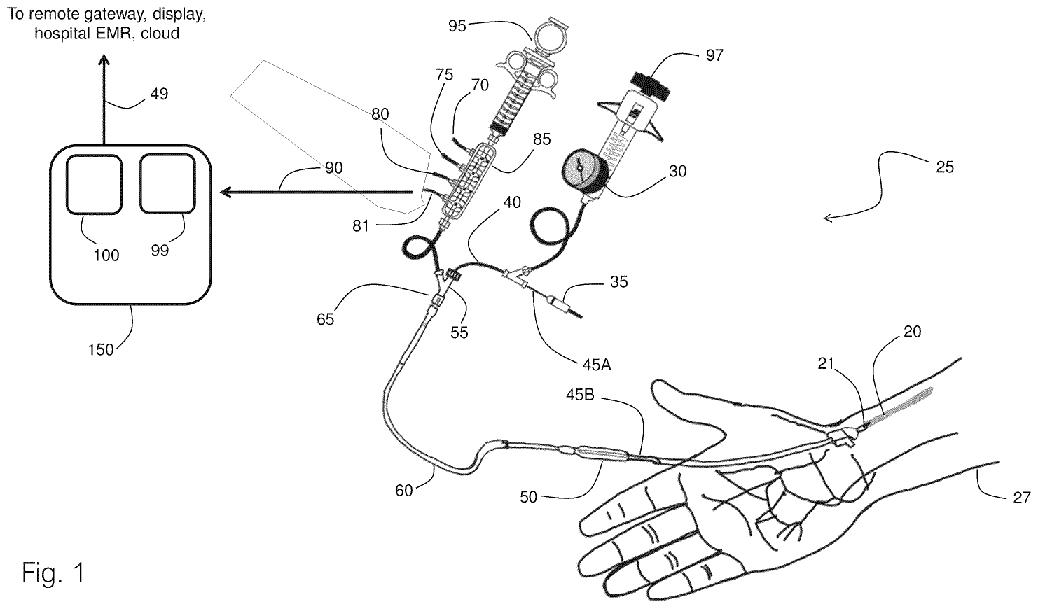

FIG. 1 shows equipment 25 used by a cardiologist during a conventional PCI procedure in a Cath Lab featuring an SSMD 150 that automatically extracts blood from a patient, and then measures ACT, H&H, and other blood-based compounds according to the invention. More specifically, the SSMD 150 includes a blood-extraction component 99 that episodically removes small volumes (e.g. <1 cc) of blood from a patient, and a measurement component 100 that measures the removed blood to determine levels of the above-described compounds using a combination of optical, electrical, and mechanical techniques, described in detail below. Taken in combination, the blood-extraction 99 and measurement 100 components monitor multiple critical hematological parameters during interventional and surgical procedures (e.g. PCI) and post-surgery recovery in a manner that is quasi-continuous, real-time, and automated.

The SSMD 150 is particularly directed towards patients receiving heparin, a ubiquitous anti-coagulant used in procedures like PCI and TAVR to prevent blood clots. Heparin is typically dosed based on the patient's weight. Through the SSMD's quasi-continuous measurements of compounds related to blood coagulation—and particularly ACT and PTT—clinicians can maintain a patient in a ‘therapeutic window’ and thus reduce the probability of deleterious clots and bleeds. The SSMD 150 potentially replaces standard POC devices and measurements conducted in a hospital's hematology lab; these episodic events require manual operation, can take hours to perform, and typically return values for just one parameter.

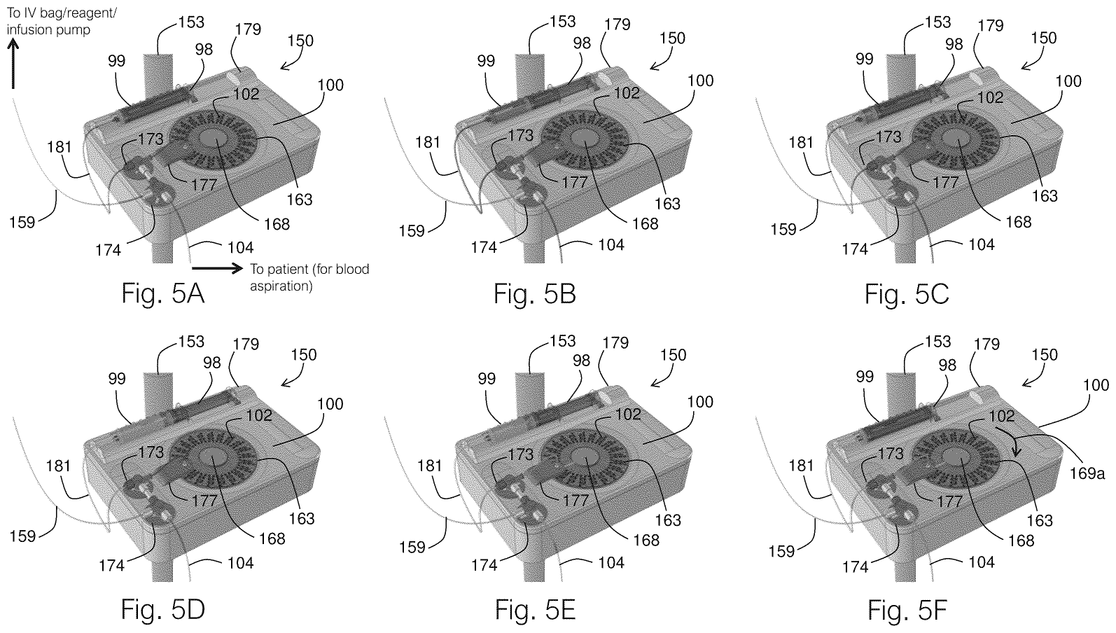

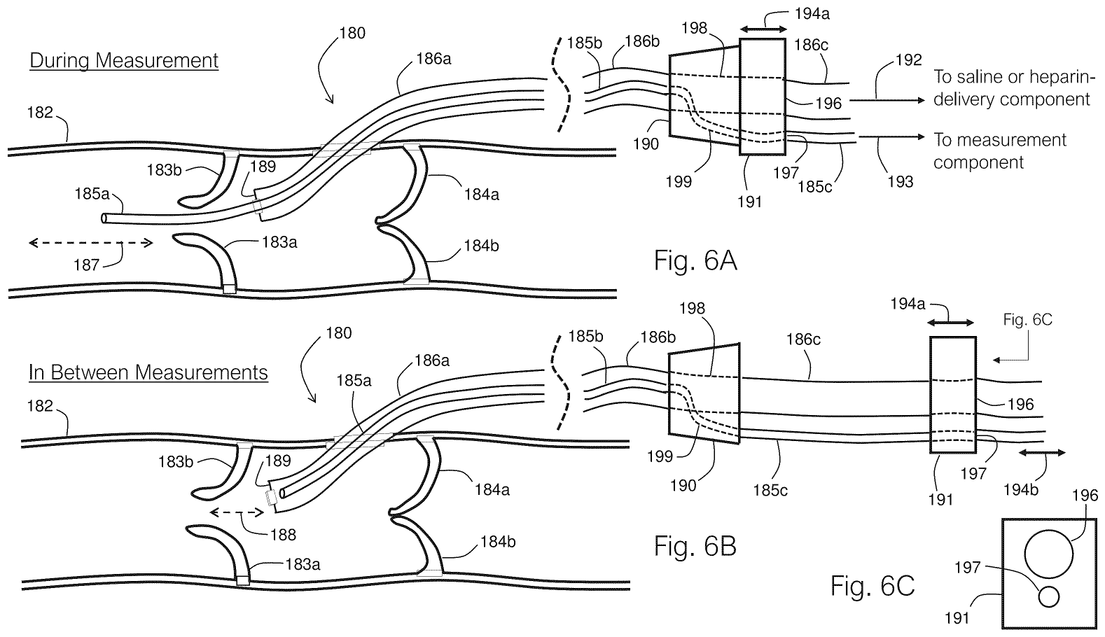

The blood-extraction component 99 in FIG. 1 features a pumping mechanism to aspirate blood (as shown in more detail in FIGS. 5A-5F, 6A-6C) that integrates with a port 81 disposed on a patient-connected manifold 85. In the embodiments shown in FIGS. 5A-5F, the pumping mechanism features a syringe 98 coupled to a linear actuator 179, effectively forming a computer-controlled ‘syringe pump’ that, during a measurement, automatically siphons off small volumes of blood from the patient. Referring back to FIG. 1, as indicated by the arrow 90, the blood-extraction component 99 removes small volumes of blood from the patient, and then automatically ports this to the measurement component 100, where, as described in detail below, it is then measured with the various optical, electrical, and mechanical sensors therein. Once a measurement is made, as indicated by arrow 49, wireless transmitters (typically Bluetooth®, Wi-Fi, or cellular radios: not shown in the figures) within the SSMD 150 transmit numerical values corresponding to these parameters, or time-dependent waveforms used to calculate them, to one of several different endpoints for further analysis, e.g. a remote gateway for display, a hospital's EMR system, or to a cloud-based software system.

During a PCI procedure the SSMD 150 measures blood aspirated from the radial artery 20 of a patient 27. Alternatively, blood may be extracted from a vein, as described in more detail below. Blood in the artery is pressurized according to the patient's arterial blood pressure (e.g. systolic, diastolic, and mean pressures), and upon insertion of an in-dwelling catheter 21, blood flows from the artery 20 into a guiding catheter 60, and from there through an arterial sheath 65 to the manifold 85. Blood passes through the manifold 85 and port 81 into the SSMD's blood-extraction component 99. Other ports 70, 75, 80 within the manifold 85 can connect to other syringes or devices (not shown in the figure), e.g., to inject into a patient a contrast agent for imaging vessels during a procedure (e.g. port 80), inject saline or other compounds (e.g. port 75), or to dispose of waste (e.g. port 70). One of the ports may also connect to a pressure transducer and from there to a vital sign monitor (not shown in the figure) that, collectively, record time-dependent arterial waveforms indicating the patient's systolic, diastolic, and mean arterial blood pressures. The cardiologist can also use a syringe 95 to administer medication that flows through the manifold 85 and into the radial artery 20, and eventually into the patient 27.

The guiding catheter 60 additionally attaches to a Y-adapter 55, which helps to seal off and prevent fluid loss, and additionally connects to the arterial sheath 65. The arterial sheath 65 passes through the guiding catheter 60 and into the patient's radial artery 20. The cardiologist uses a coronary guidewire 45A, 45B to assist with advancing the guiding catheter 60 through the Y-adapter 55 and into the arterial sheath 65. A guiding system 35 controls the coronary guidewire 45A, 45B and guiding catheter 60. Once the arterial sheath 65 is inside the radial artery 20, the cardiologists uses the guiding system 35 to manipulate the guiding catheter 60 and advance a balloon catheter 40 connected to it towards the patient's heart. An inflation system 97 inflates the balloon catheter 40, and additionally connects to a pressure sensor 30 that measures the pressure within therein. During the procedure, the cardiologist uses the guiding system 35 to pass the balloon catheter 40 over the guiding catheter 60 to the artery identified as having a blockage. The cardiologist then uses the inflation system 97 to inflate a balloon 50 located at the tip of the balloon catheter 40. The inflating balloon 50 presses against plaque in the coronary artery to push it against a wall of the artery it is disposed in. The cardiologist may inflate the balloon 50 several times depending on the procedure. A stent (not shown in the figure) can be placed at the tip of the balloon catheter 40, over the balloon 50. In this case, during the procedure, the cardiologist uses the guiding system 35 to guide the balloon catheter 40 to the site of the blockage, and inflates the balloon 50 to expand the stent. After the stent is deployed within the artery, the cardiologist deflates the balloon 50 and, using the balloon catheter 40 and guiding catheter 60 controlled by the guiding system 35, removes the balloon 50 from the patient.

### 2. SSMD Measurement System

Throughout this procedure, the SSMD 150 aspirates blood and then measures certain compounds therein. To do this, the blood-extraction component 99 removes blood from the patient using a system similar to that shown in FIGS. 5A-5F, 6A-6C, and then ports the blood sample to the measurement component 100 to measure ACT, H&H, and other blood-based compounds.

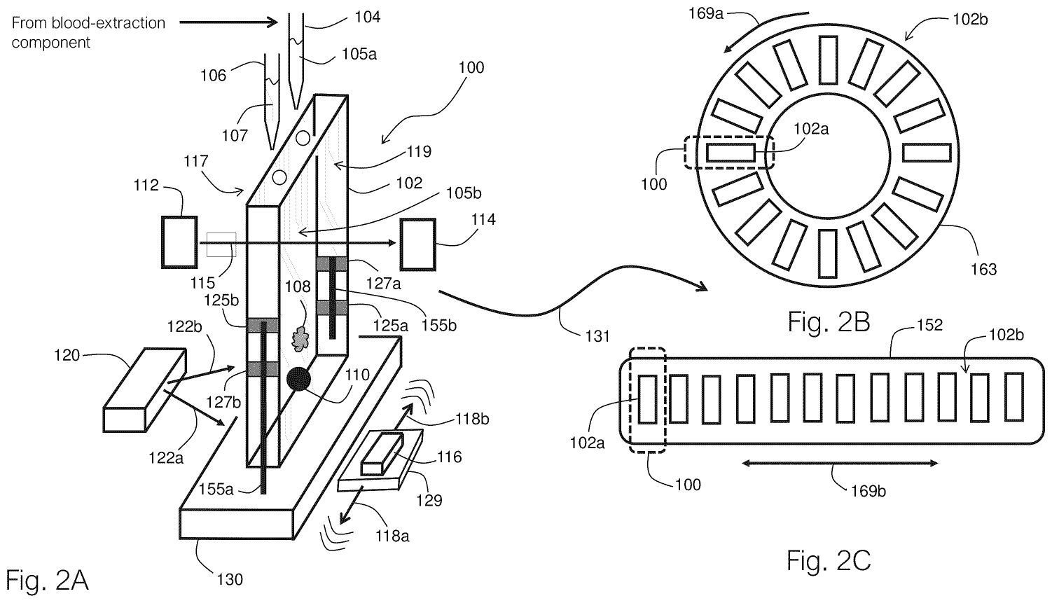

More specifically, as shown in FIGS. 2A-C, the measurement component 100 features a measurement cell 102 designed to make optical, electrical, and mechanical measurements from a sample of blood 105b from the patient. An automated pipetting system 104, or similar mechanism, receives blood 105a from the blood-extraction component 99 and automatically deposits it into the measurement cell 102 so that a measurement can be made. In embodiments, a second pipetting system 106 (or, again, a similar mechanism) deposits a chemical reagent 107 in the measurement cell to facilitate a measurement. The reagent 107, for example, may be a chemical compound that reacts with a compound in the blood 105b. Examples of this include a reagent that features an aptamer that specifically binds to a clotting factor in the blood (e.g. a protein, such as thrombin). The aptamer may be coupled to an optically measurable compound, such as one that exhibits a colorimetric change. Aptamers are short sequences of artificial DNA, RNA, XNA, or peptide that bind a specific target molecule, or family of target molecules. They exhibit a range of affinities (KD in the pM to μM range) with little or no off-target binding and are sometimes classified as chemical antibodies'. Aptamers and antibodies can be used in many of the same applications, but the nucleic acid-based structure of aptamers, which are mostly oligonucleotides, is very different from the amino acid-based structure of antibodies, which are proteins. This also means aptamers can be made quickly and at low cost with commercially available DNA sequencers.

During optical measurements made by the measurement component 100, the magnitude of colorimetric change, as measured by one of the optical systems described in detail below, indicates the concentration of the targeted protein in the blood. In related embodiments, the measurement cell 102 may include a solid clotting agent 108, typically in the form of a powder, that mixes with the blood 105b to stimulate clotting. The clotting agent 108, for example, can be diatomaceous earth (i.e. kaolin or clay), thrombin, or something similar that reacts with fibrinogen, a blood clotting factor that is normally dissolved in blood, to form long strands of fibrin that radiate from the clumped platelets and form a net that entraps more platelets and blood cells during clotting.

For H&H measurements, the clotting agent 108 may also contain sodium deoxycholate, which lyses red blood cells and causes the release of hemoglobin. In the presence of added sodium nitrite, the released hemoglobin converts to methemoglobin. Methemoglobin, when mixed with sodium azide, yields azide methemoglobin. This sample can be measured at two wavelengths using the optical system described herein: 1) 570 nm to determine methemoglobin and 2) 880 nm to make up for sample turbidity.

Hematocrit is the volume percentage of red blood cells in blood. Its measurement depends on the number and size of red blood cells, and is normally 40.7-50.3% for males and 36.1-44.3% for females. Calculated hematocrit is typically determined by dividing the red cell volume by the total volume of the blood sample. A typical measurement of hematocrit involves centrifuging EDTA-treated or heparinized blood in a capillary tube (also known as a microhematocrit tube) at 10,000 RPM for about five minutes This separates the blood into layers. The volume of packed red blood cells divided by the total volume of the blood sample gives the hematocrit. With the measurement cell 102, this can be calculated by measuring the lengths of the layers using, e.g., the small-scale camera and image-processing software described herein.

To measure a colorimetric change in the blood sample, a light source 112 and optical photodetector 114 forming an optical system may be coupled to the measurement cell 102. Measurements with this system yield an optical property such as absorption or scattering of blood 105b within the cell. During this type of measurement, optical radiation from the light source 112 passes through a first optically transparent window 117, through blood 105b in the measurement cell 102, through a second optically transparent window 119, and into the photodetector 114, as indicated by arrow 115. The blood 105b, in turn, will absorb the incident radiation according to its absorption spectrum, and also scatter it; both these processes evolve over time as the blood clots.

The light source 112 may be a laser, narrow-band light emitting diode (herein “LED”), or broadband LED, i.e. a source of ‘white light’ spanning the ultraviolet, infrared, and visible spectral ranges. The photodetector 114 may be a conventional photodiode that converts incident optical radiation into an electrical current that then passes through a resistor or transimpedance amplifier to yield a voltage, which can then be measured with an analog-to-digital converter to generate a time-dependent optical signal, such as that shown in FIG. 17B. As shown in this figure, the time-dependent optical signal represents the amount of radiation emitted from the light source 112, as indicated by the arrow 133, that passes through the transparent optical window 117, and into blood 105b within the sample cell 102. There, the radiation is partially absorbed and scattered by the blood 105b, as described above. The combination of these two factors-time-resolved optical absorption and scattering-decreases the intensity of radiation that is incident on the photodetector 114. This results in a time-dependent signal that gradually decreases in intensity, as shown in FIG. 17B. As shown, at a time consistent with the ACT, as indicated by arrow 138, the blood rapidly clots; this rapid liquid-to-solid phase transition dramatically increases the amount of light scattering, thus causing a rapid drop in the amount of light that reaches the photodetector 148.

In related embodiments, the photodetector 114 may be a spectral detector, such as the AS7262, AS7341, AS7343, or AS7421 chip-based solutions, all marketed by AMS Inc. (see, e.g., https://ams.com/en/spectral-sensing). These detectors are incorporated into a small-scale package (typically about 3×3 mm) and feature a sensitive photodiode positioned behind a set of miniaturized, computer-controlled Fabry-Perot etalons that act as programmable optical filters. During use, a computer program operating on a microcontroller within the SSMD and coupled to the AMS spectral detector sets a specific register, which in turn activates a specific optical filter that passes a narrow bandwidth of incident optical radiation (e.g., from λ=600-630 nm). The photodiode associated with the detector detects the narrow-band radiation in this region for a short period of time (e.g. a few milliseconds), generates a digital signal (i.e. a number of ‘counts’) that the microcontroller receives through a serial port. After this the computer program sets a new register that passes a new, narrow bandwidth of optical radiation (e.g., from λ=630-660 nm), and the process is repeated until a series of discrete signals spanning an optical spectrum is collected, with the series representing an optical absorption spectrum of the blood 105b within the sample cell 102. The series of discrete signals serves as a proxy for a complete absorption spectrum, which is typically measured with a much larger (and relatively expensive) apparatus, such as an absorption spectrometer featuring a tungsten light source, diffraction grating, and CCD camera. FIG. 19, for example, also shows such an absorption spectrum (continuous trace), as measured by an absorption spectrometer marketed by Thorlabs Inc. (https://www.thorlabs.com/newgrouppage9.cfm?objectgroup_id=3482). Discrete signals (individual triangles) measured by the AS7262 part, which sequentially measures six narrow bandwidths of optical radiation ranging from about λ=450-650 nm, each represented by an individual triangle in the plot. In FIG. 19 the two spectra are overlaid, indicating how the signals measured with the AS7262 part, while discrete, include comparable information to that determined with the Thorlabs spectrometer. The discrete data points measured by the AS7262 part can be ‘filled in’ using a computer algorithm, such as an interpolation or spline algorithm, to recreate the continuous spectrum measured with the conventional spectrometer.

Using this approach, for example, an algorithm operating on the microcontroller within the SSMD can determine the amount of hemoglobin in a blood sample, as described in more detail below with reference to FIGS. 18A and 18B. As discussed above, prior to this measurement, the blood sample may be processed with a clotting agent that includes contain sodium deoxycholate, to lyse red blood cells and drive the release of hemoglobin, which is then probed with optical measurements near λ=570 nm.

Moreover, as blood clots its optical absorption spectrum changes. Thus, in embodiments, the optical absorption spectra measured with the AMS part or comparable optical spectrometer, or alternatively time-resolved optical signals measured with a conventional photodetector, yield time-resolved signals that can be processed to determine ACT, PTT, or other coagulation parameters that relate to time-dependent clotting of blood.

In a related embodiment, the light source 112 and photodetector 114 detect time-resolved optical scattering, e.g. scattering driven by blood clotting in the sample cell 102. In this case, the ideal light source is a laser, focused onto the sample cell using a first lens to collimate diverging light from the laser, and a second lens to focus the collimated light onto the sample cell 102. Light scattering increases as blood clots and undergoes a transition from a liquid to solid phase, with a portion of the scattered light refracted so that it is not incident on the photodetector 114. This decreases signals measured by the photodetector in a time-dependent manner that, once analyzed, yield ACT. Here, the time-dependent signal will look similar to that shown in FIG. 17B. In a related embodiment, the photodetector 114 can include a spatial array of photodiodes, with those removed from a center portion of the array placed to detect clot-induced scattered radiation. Here, a time-dependent increase in signal levels measured by these distal photodiodes, once analyzed, yields ACT.

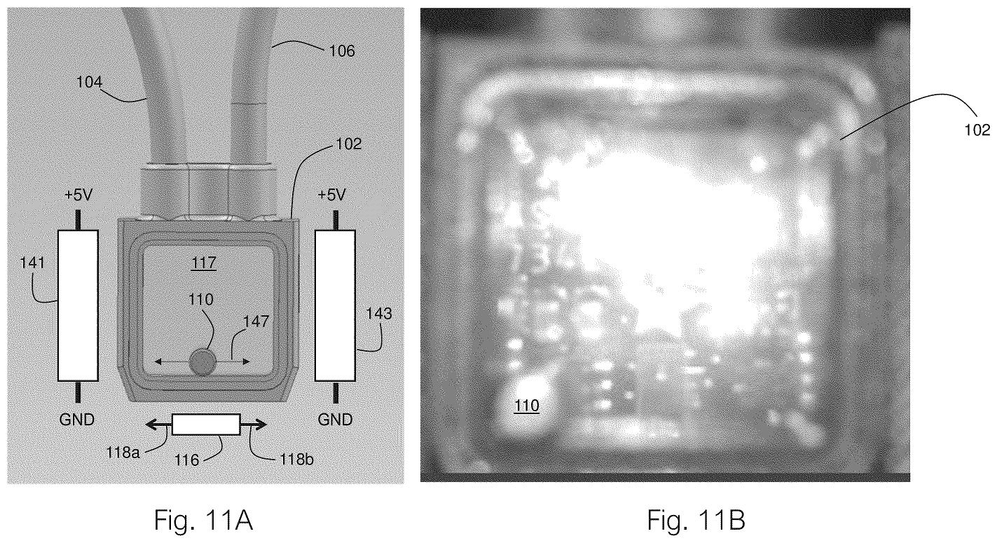

Referring again to FIG. 2A, an optical imaging technique can also be used to measure clotting blood in the measurement cell 102. In this embodiment, the cell 102 includes a metal ball 110 composed of a magnetically active material (e.g. iron). A magnet 116 disposed on a linear actuator 129 rests underneath a base 130 in the measurement system 100. During a measurement, a microcontroller coupled to a servo motor (not shown in the figure) moves the linear actuator 129 and the overlying magnet 116 back and forth along a span of the measurement cell 102, as indicated by the arrows 118a, 118b. This, in turn, causes the metal ball 110 within the cell to move back and forth in a consistent manner, with the motion impeded over time due to the viscosity increase and phase transition of the clotting blood. A small-scale video camera 120, with a field of view indicated by arrows 122a, 122b, records time-dependent images of the magnetically translating metal ball 110. FIG. 11B show an example of such an image; this was collected with a video camera and measurement cell similar to that shown in FIG. 2A.

As blood clots within the cell, movement of the metal ball 110 is gradually impeded, and the video camera 120 records a video of this motion. Processing the video with an image-processing algorithm yields a two-dimensional (or, in embodiments, three-dimensional) time-dependent trace indicating motion of the metal ball 110. The time-dependent trace will reflect a back-and-forth movement of the metal ball 110 within the cell wherein the distance that it travels within the cell is gradually reduced, and eventually ceases completely, when all the blood within the cell is fully clotted; in this way, for example, the trace may resemble that of a damped harmonic oscillator.

Processing of the two-dimensional (or three-dimensional) trace determined from multiple time-dependent images, each like that shown in FIG. 11B, may involve sophisticated algorithms, such those involving artificial intelligence (herein “AI”) and/or machine learning (herein “ML”), to extract and analyze information for this mechanical measurement. In one embodiment, once the time-dependent trace is extracted, it may be analyzed with numerical ‘fitting’ techniques that iteratively vary parameters of a ‘fitting function’ until the function best matches the time-dependent trace. Here, ‘best matching’ is typically determined with a fitting parameters called ‘χ²’ which represents a minimum error between the fitting function and the actual data. For example, if the trace does indeed reflect the motion of a damped harmonic oscillator, the fitting function may include an exponential damping component (e.g. e^{-γt}, where γ is a damping constant and t is time) multiplied by a sine wave (e.g. sine(ωt)), where t is again time and ω is a frequency corresponding to the oscillatory nature of the metal ball 100 driven by the magnet 116 within the measurement cell 102. Here, ω is determined by the periodic movement of the linear actuator 129 and magnet 116, and is thus a known quantity; γ indicates clotting time and, thus, ACT.

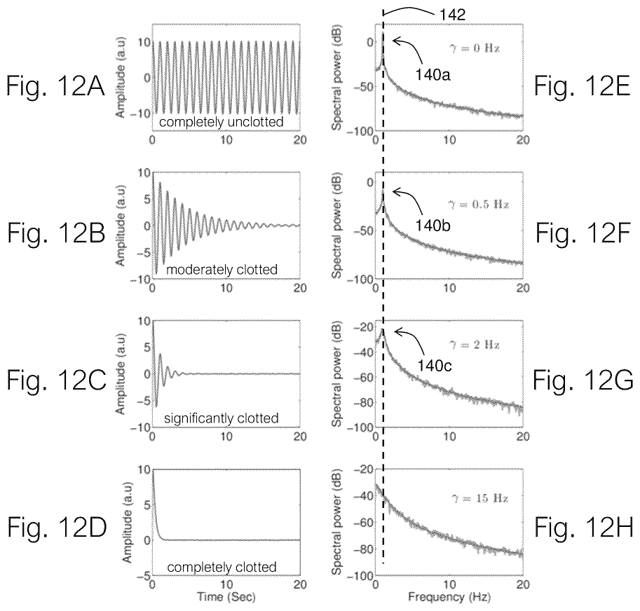

FIGS. 12A-12D show specific examples of time-domain signals that indicate this physiology and the fitting process used to model them and thus predict ACT; these are described in more detail below.

In related embodiments, the time-dependent linear trace described above may be processed with a Fourier Transform, or similar mathematical operation, to transform the time-dependent trace into the frequency domain. For example, FIGS. 12E-12H show corresponding Fourier Transforms of the time-domain waveforms in FIGS. 12A-12D. Such frequency-domain waveforms may make it easier to extract the requisite parameters from the data. For example, the Fourier Transform of the time-domain data shown in FIGS. 12A-12D is best described by a Gaussian function (ƒ(t)˜e^{-γt} sin(ωt)), where ω is the central frequency of the function and γ/2 characterizes its width.

The metal ball 100, when magnetically coupled to the magnet 116 and linear actuator 129, may also be used to mix blood 105b within the measurement cell 102 with other components. For example, this process can be used to mix blood with the clotting agent 108 or a chemical reagent 107. Here, at the beginning of a measurement, the microcontroller and servo motor controls the linear actuator 129 and attached magnet 116 to rapidly move back and forth. This action, in turn, will cause the magnetic ball 110 to rapidly move back and forth within the measurement cell 102 to mix the blood 105b with the clotting agent 108 or a chemical reagent 107. Once the mixing is complete (this could be detected by the camera, for example, or simply be conducted for a set time period), the measurement process begins, as described herein.

In related embodiments, the video camera 120, alone (i.e. without using the magnetic ball 110, magnet 116, or linear actuator 129) can image blood 105b within the measurement cell to detect clotting blood. For example, when blood 105b in the measurement cell 102 clots, its general appearance will gradually change, and can thus be detected by collecting time-resolved images with the video camera 120, and then analyzing these with image-processing algorithms (including, e.g., algorithms based on AI and/or ML) to determine parameters such as ACT.

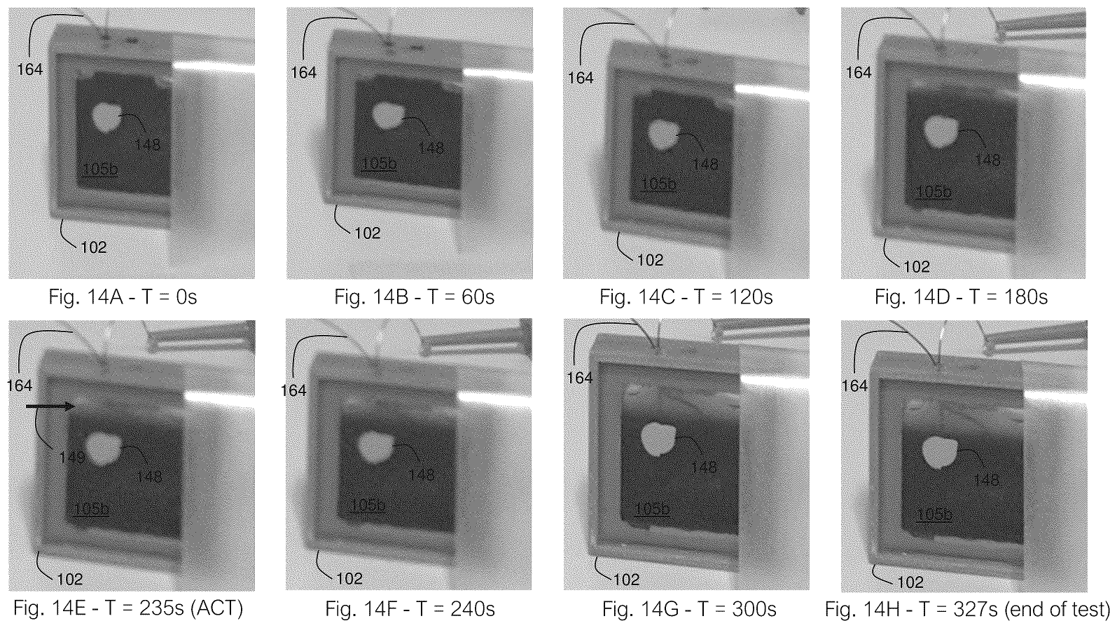

FIGS. 14A-14H show this, as an example. Here, clotting blood 105b within the sample cell 102 was measured with both imaging and impedance techniques. Images in these figures were collected from blood samples aspirated during a PCI procedure. Once aspirated, blood 105b was ported to the measurement cell 102, which included transparent optical windows coated on their inner surface with a thin film of ITO, a material that is both optically transparent and electrically conductive. To make an impedance measurement, the cell 102 features a set of conductive wires 164 that connect an impedance circuit (not shown in the figure) to a dab of conductive epoxy 148, which in turn is bonded to the ITO film. Both windows in the optical cell were fabricated in this way. Sense and drive electrodes associated with the impedance measurement are shorted together.

Measurements made in the cells were made over a period of 327 seconds, and indicate how the appearance of blood, which was mixed with kaolin prior to the measurement, changes as it gradually clots over time. The arrow 149 in FIG. 14E, for example, indicates a portion of the image showing an increase in clotting blood; this time corresponds to ACT, as measured with an external POC device and indicated in the figure.

In a related embodiment, the video camera can detect colorimetric changes of blood 105b within the measurement cell. Such a measurement would occur, for example, when a reagent 107 dispensed from the pipette 106 is mixed with blood within the measurement cell 102. Similar to the case described above, in embodiments the reagent 107 is a chemical compound (e.g. an aptamer) that specifically binds to clotting factors (e.g. thrombin) in the blood. To indicate a colorimetric change, the reagent is typically coupled to a molecular compound with a distinct colorimetric signature, e.g. one that could be detected by the video camera 120. The intensity of the colorimetric change detected by the camera 120 relates to the concentration of the clotting factors in the blood.

In embodiments, the measurement component 100 additionally includes a centrifuge that ‘spins down’ the measurement cell 102 and the blood therein to separate blood cells (e.g. leukocytes, thrombocytes, and erythrocytes) from plasma. Here, the measurement cell 102 is spun down prior to making any measurements with the sensors described herein. The camera 120 can then image the centrifuged measurement cell 102 to measure hematocrit, which is the volume percentage of red blood cells in the blood sample. Here, the image is processed to detect the separate layers in the centrifuged cell: plasma is relatively clear, while leukocytes and thrombocytes have a white/gray color, and erythrocytes are deep red. The microprocessor in the measurement component determines hematocrit by detecting the separated components and calculating the ratio between erythrocytes and the total blood sample. Because the purpose of red blood cells is to transfer oxygen from the lungs to body tissues, a blood sample's hematocrit—the red blood cell volume percentage—indicates the blood's capability of delivering oxygen to vital organs in the body. Hematocrit levels that are too high or too low can indicate a blood disorder, dehydration, or other medical conditions. An abnormally low hematocrit may suggest anemia, a decrease in the total amount of red blood cells, while an abnormally high hematocrit (polycythemia) may indicate a relatively high probability of heart attack, stroke, pulmonary embolism, and/or enlargement of the liver and spleen.

Centrifuging the measurement cell separates plasma from blood cells. In embodiments, a measurement component that includes a centrifuge may also include a system for selectively extracting the plasma after centrifuging so follow-on tests (e.g. for PTT) can be made on this medium. In general, PTT is assumed to be a more accurate measurement of clotting compared to ACT, hence its use as a coagulation metric for patients on heparin during post-surgery recovery. PTT is not typically measured in the Cath Lab, mostly because of time required to complete the test, and convenience: during procedures like PCI, it is paramount to get results as quickly as possible, and this is best done with ACT.

To extract plasma from the centrifuged blood sample, the measurement component may include an additional pipetting system (e.g. one similar to component 104 and 106 in FIG. 2A) that extracts the plasma and deposits it in a new measurement cell (e.g. a new cell in the circular or linear array of cells, as shown in FIGS. 2B and 2C, respectively).

For a PTT test, the measurement cell is first centrifuged to separate blood cells from plasma. The plasma is then extracted from the measurement cell as described above, and a clotting agent (such as silica, celite, kaolin, diatomaceous earth, ellagic acid, clay) is added to the sample. This activates the intrinsic pathway of coagulation, and the time the sample takes to clot is measured optically, as described herein. Once deposited in the new cell, the assay can take place.

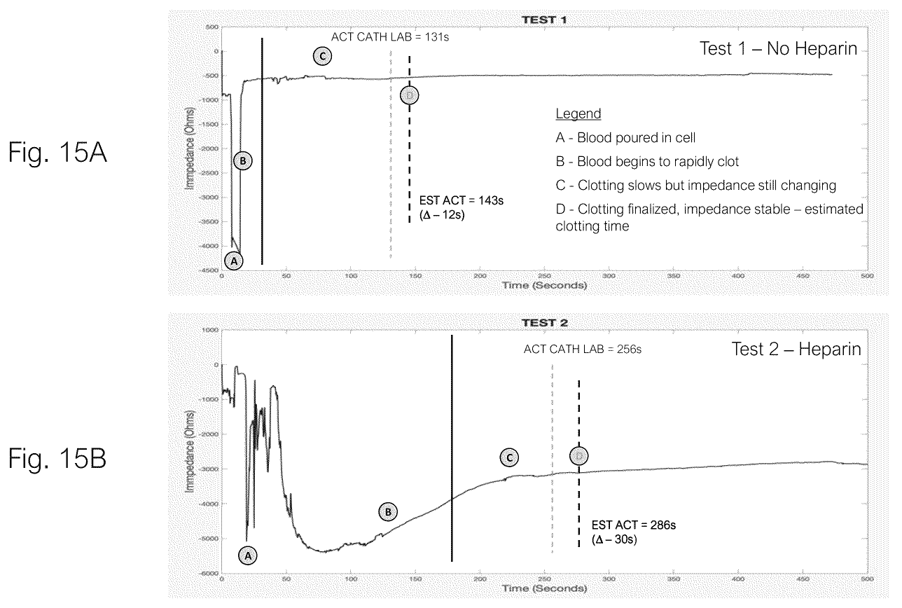

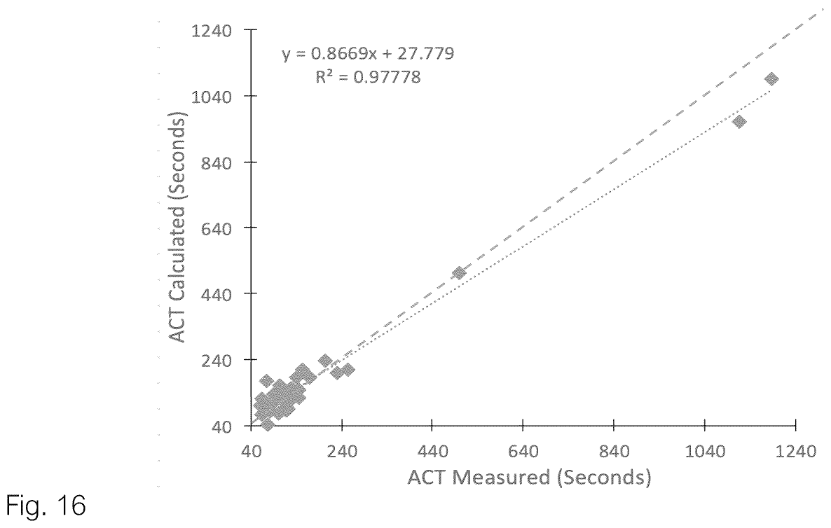

An electrical measurement, such as electrical impedance or reactance (which detects phase changes of electrical current flowing through the blood sample), can also be used to detect clotting blood in the measurement cell 102. In a clinical study investigating this, measurement cells similar to those in shown in FIG. 14 were used to measure time-dependent impedance waveforms like those shown in FIGS. 15A and 15B. These data were then analyzed along with corresponding measurements from a POC reference device to yield the scatter plot shown in FIG. 16.

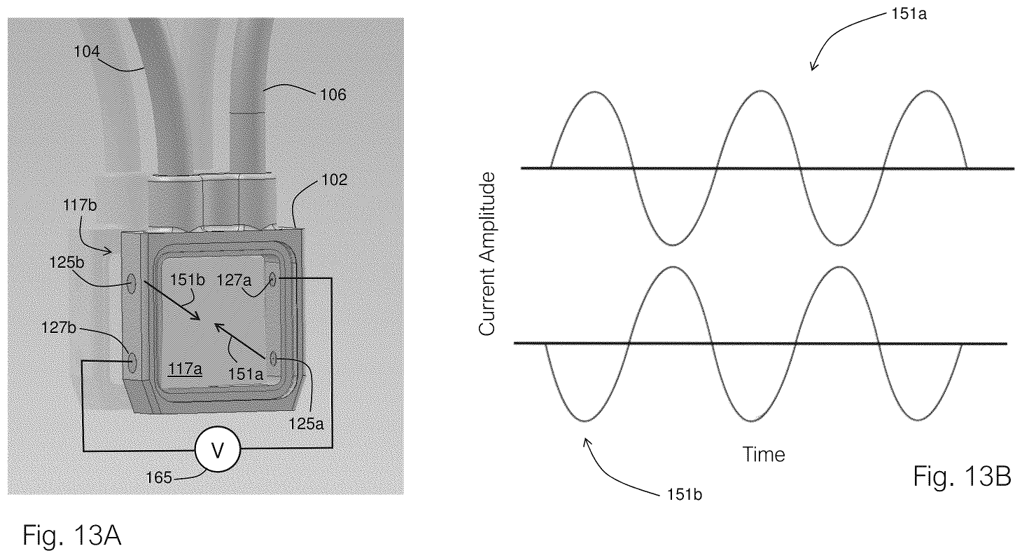

Referring again to FIG. 2A, to perform such a measurement the sample cell 102 includes pairs of sense 125a, 125b and drive 127a, 127b electrodes that connect to electrical connectors 155a, 155b on both sides of the measurement cell. In embodiments, for example, the electrical connectors 155a, 155b are retractable and conductive ‘pogo pins’, or something comparable, that press into the sense 125a, 125b and drive 127a, 127b electrodes when the measurement cell is inserted into the base 130. Typically, the sense 125a, 125b and drive 127a, 127b electrodes are metal rods that extend into the measurement cell 102 so that they contact the blood therein. The electrical connectors 155a, 155b connect to an impedance/reactance circuit (not shown in the figure) disposed in the base 130 of the measurement system 100. During a measurement, and as indicated by FIGS. 13A and 13B, the drive electrodes 125a, 125b inject high-frequency (e.g. 5-500 kHz), low-amperage (e.g. 0.1-4 mA) alternating current (herein “AC”) into the measurement cell, with the AC from opposing drive electrodes being 1800 out of phase (as indicated in FIG. 13B). The sense electrodes 125a, 125b detect the electrical impedance encountered by the injected current, and in response yield a potential difference between these two terminals that can be amplified with a differential amplifier and then digitized with an analog-to-digital converter, both of which are incorporated in the impedance/reactance circuit.

In embodiments, the injected drive current, as shown in FIG. 13B, can feature a time-dependent profile other than a sine wave. For example, the profile can be a square or triangular wave.

For these impedance measurements, the AC injected by the drive electrodes 127a, 127b current can either be at a constant frequency (e.g. 100 kHz) or, in some embodiments, the impedance circuit rapidly ‘sweeps’ the AC frequencies between frequency ranges (e.g. from 5-1000 kHz). Sweeping the frequency allows the measurement component to make spectroscopic measurements. Technically, the impedance measured with the circuit is a complex term, wherein electrical resistance encountered by the electrical current represents the real component of the impedance, and reactance encountered by the current represents the imaginary component of the impedance. More specific to this particular measurement, impedance (the real component of the signal) is typically impacted by the electrical conductance of blood within the measurement cell (i.e. resistance), whereas reactance (the imaginary component of the signal) is typically impacted by the electrostatic storage of charge (i.e., capacitance) of the blood.

Both resistance and capacitance of the blood will change in a tine-dependent manner as it clots. FIGS. 15A and 15B indicate changes in electrical impedance, as measured with a measurement cell and impedance/reactance circuit similar to that shown in FIG. 2B, for blood containing no heparin (which clots very fast, as shown in FIG. 15A) compared to blood lacking heparin (which clots very slowly, as shown in FIG. 15B). These waveforms represent the low frequency “DC components” of the impedance signal. As shown in both figures, when blood, a highly conductive medium, is poured into the cell, its impedance drops rapidly (indicated by ‘A’). Blood that is not heparinized clots fast, causing its impedance to rapidly increase (‘B’ in FIG. 15A), whereas heparinized blood clots much more slowly (‘B’ in FIG. 15B). This clotting becomes more gradual over time (‘C’ in both figures), with the clotting eventually stabilizing, yielding an ACT (‘D’ in both figures). For this study, ACTs measured with this approach were compared to those measured with a conventional POC device, as indicated by ‘ACT Cath Lab’ in the figure. The impedance-measured data agreed with the POC-measured data to within 12 and 30 seconds for the data shown in, respectively, FIGS. 15A and 15B.

FIG. 16 shows a scatter plot indicating results from a clinical study conducted using data similar to that shown in FIGS. 15A and 15B. Here, the v-axis (‘Calculated Impedance’) shows ACT values calculated using time-dependent waveforms measured by the impedance sensor and algorithmic approach indicated in these figures. The x-axis shows ACT measured with a conventional POC device during a PCI procedure. As indicated in the figure, ACT values determined with these respective approaches have strong agreement (r=0.977), indicating the efficacy of the impedance approach.

Additionally, as blood clots it changes the capacitance of the measurement cell, as measured between the two sense electrodes 125a, 125b. The capacitance of the measurement cell can be measured by sweeping the frequency of the injected current, and observing a rapid change in reactance, which in turn indicates a resonant frequency within the cell. The resonant frequency, in turn, can be analyzed to determine the capacitance of the cell, with these two parameters—resonant frequency and capacitance—being inversely related. Analyzing these parameters as a function of time can indicate ACT.

In related embodiments, the first 117 and second 119 optically transparent windows include isolated films of ITO. Each transparent window 117, 119 includes two regions of ITO, with one serving as a sense electrode, and the other a drive electrode. The regions of ITO replace the metallic sense 125a, 125b and drive 127a, 127b electrodes described above, and can be used to measure impedance and reactance signals, as described above.

Referring to FIGS. 2B and 2C, and indicated by the arrow 131, sets of measurement cells can be incorporated into a measurement component 100 featuring a circular array 163 of cells (FIG. 2B), or a linear array 152 of cells (FIG. 2C). For example, as shown in FIG. 2B, the circular array 163 includes a collection of measurement cells 102b disposed within the array 163. These cells 102b can be identical (e.g., each cell may contain a solid clotting agent 108 like that shown in in FIG. 2A). Alternatively, the measurement cells 102b may be different, e.g. each cell may include a different set of components that facilitate a different measurement, e.g. a unique ion-specific electrode designed to pass a certain ion within the blood sample (e.g. potassium or chlorine ions). In related embodiments, one or more of the cells may include amperometric systems, e.g. amperometric glucose biosensors. These are prepared by immobilizing glucose oxidase molecules onto an electrochemical interface within the measurement cell. When exposed to blood, this enzyme catalyzes the conversion of glucose to gluconic acid and hydrogen peroxide. Glucose is quantified by the electrochemical measurement of hydrogen peroxide through a typical ‘redox reaction’. More specifically, current generated during this reaction passes through a circuit associated with the electrochemical interface, and is converted into a voltage with a transimpedance amplifier. The processing system digitizes the voltage and converts it into a glucose level using an algorithm, such as a simple look-up table that relates these two parameters.

In still other embodiments, all the measurement cells 102b within the array 163 may be identical to each other, but during a measurement they may receive different reagents 107 through the pipette 106, thus initiating a different chemical reaction that can be analyzed with the optical, electrical, and mechanical sensors described above to determine a unique compound within the blood. These same cells 102b, and the same measurement methodology, may be used for the linear array 152 shown in FIG. 2C. Sets of measurement cells disposed in other geometries can be used according to the invention.

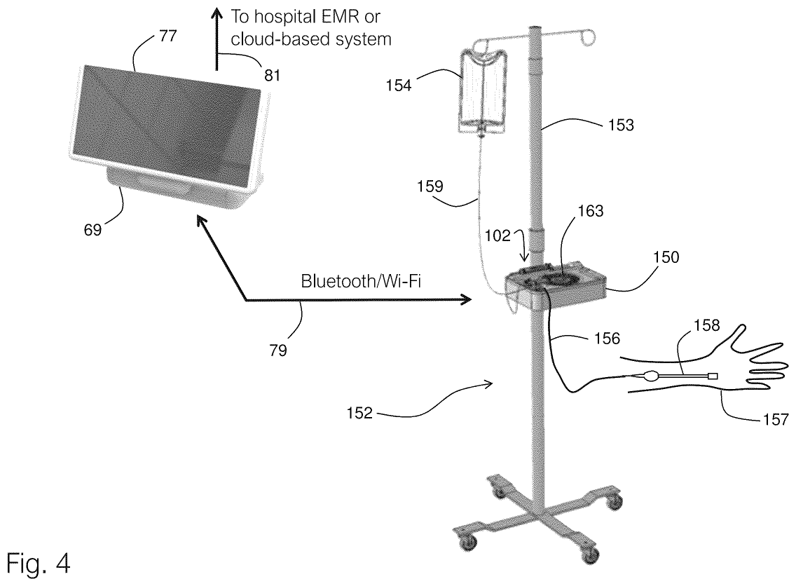

By including sets of measurement cells, the measurement components 100 shown in FIGS. 2B and 2C allows a collection of measurements to be made during a medical procedure, e.g. a PCI procedure. For example, prior to the procedure, a clinician may operate a user interface running on a touchpanel display (e.g. a touchpanel display 77 shown in FIG. 4) to ‘program’ a measurement sequence into the SSMD 150. The measurement sequence may include the types of measurements the clinician desires to make during the procedure, along with how often they want to make the measurements (e.g. ACT measurements every 15 minutes, H&H measurements every 60 minutes, potassium and chloride ion measurements made at the very beginning of the procedure, a PTT made at the very end of the procedure). Once this is programmed into the SSMD 150, the microcontroller that controls the blood-extraction and measurement components can control a servo component (not shown in the figure) to move the circular array 163 in a rotational manner as indicated by arrow 169a, or the linear array 152 in a linear translational manner as indicated by arrow 169b, to position the appropriate cell 102a proximal to the measurement component 100. The measurement component 100 then makes a pre-programmed measurement as described above. Once the measurement is made, a numerical value representing the appropriate parameter (e.g. ACT, H&H) is transmitted (either through a wired or wireless interface) to the touchpanel display 77, where it is shown, e.g. in a time-resolved graphical format. The servo component then rotates the circular array 163, or translates the linear array 152, to position a new measurement cell 102b proximal to the measurement component 100, and the process is repeated.

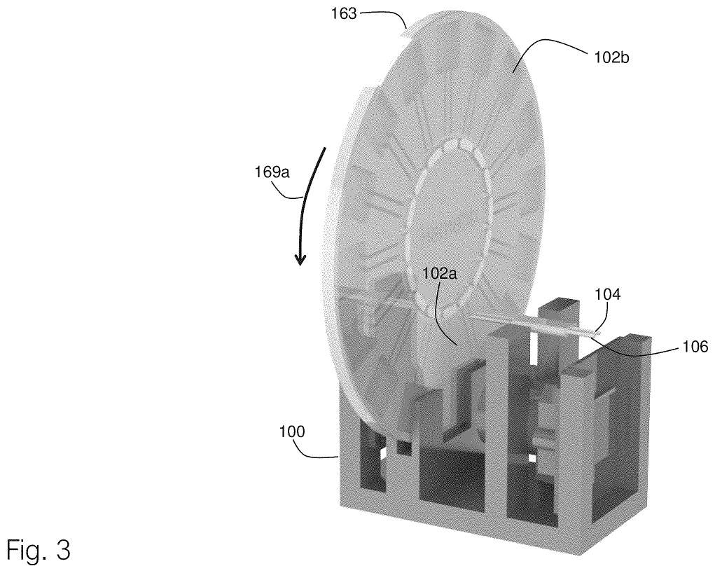

FIG. 3 shows a mechanical drawing of the circular array 163 of measurement cells 102b and a measurement component, as shown in FIG. 2B. The figure indicates how a microcontroller-controlled servo motor can rotate the circular array 163 so that the appropriate measurement cell 102a is positioned proximal to the measurement component 100. As described above, pipettes 104, 106 integrated with this system deliver, respectively, blood and reagents to the measurement cell, where they are mixed together (e.g. using the metal ball within the measurement cell, as described above) prior to the measurement.

As with all data used in the hospital, a packet used to transmit the data between the SSMD and the touchpanel display is encrypted; the user interface operating on the display follows guidelines established by the Health Insurance Portability and Accountability Act of 1996 (herein “HIPPA”).

### 3. Examples of Measurements Made During Surgical Procedures and Post-Surgery Recovery

FIG. 4 shows a fully integrated measurement system 152 that features the SSMD 150 integrated with a conventional IV pole 153. The system 152 is used, for example, during a surgical procedure or post-surgery recovery. A catheter 158 inserted into a vein within an arm 157 of a patient aspirates blood, which passes through a first flexible tube 156 and into the SSMD 150. From there, a pipette (not shown in the figure) or a similar mechanism ports the blood into a measurement cell 102 within a circular array 163 containing multiple measurement cells. Likewise, an V bag 154 attached to the IV pole 153 provides fluids and/or reagents through a second flexible tube 159 and into the SSMD 150. A touchpanel display 77 features two-way communication with the SSMD 150, as indicated by the arrow 79. Communication can be through a wired mechanism (e.g. a serial cable), but is preferably made using a wireless protocol, e.g. Wi-Fi or Bluetooth®. For example, and as described above, a customized user interface operates on the touchpanel display 77, allowing the clinician to program into the user interface a desired sequence of measurements to be made during a surgical procedure or post-surgery recovery. In alternate embodiments, the SSMD 150 can include functionality to make immediate, episodic measurements, as opposed to (or in addition to) pre-programmed periodic measurements. Here, the user interface operating on the touchpanel display 77 includes a software ‘on-demand button’ that, once clicked by the clinician, sends a code through the Wi-Fi or Bluetooth® interface to the SSMD 150, instructing it to immediately make a measurement. This can occur within seconds, after which the corresponding numerical value is transmitted back to the touchpanel display 77 for display to the clinician.

Measurements made in this manner during the surgical procedure typically focus on ACT and H&H, as these are standard for patients receiving heparin, and typically occur at relatively high frequency (e.g. every 15-30 minutes) using POC devices. Conversely, measurements made during post-surgical recovery typically focus on PTT, H&H, and other blood-based compounds, such as glucose, potassium and chloride ions, lactate and lactic acid, cortisol, and other biomarkers. These measurements, which are typically made from patients on heparin drips, usually occur at relatively low frequencies (e.g. every few hours) using blood tests in the hospital's hematology laboratory. In both cases, the SSMD 150 makes the measurements automatically without requiring any human interaction. Numerical values corresponding to measurements made by the SSMD 150 are transmitted through Wi-Fi or Bluetooth® (again, according to the arrow 79) back to the touchpanel display 77, where they are displayed to the clinician and additionally transmitted to the hospital EMR or similar cloud-based system, as indicated by arrow 81. Note that the touchpanel display 77 is typically connected to a computer 69 configured to process information that the SSMD 150 generates. For example, the computer 69 can simply plot out graphical representations of the data for the clinician, e.g. trends, histograms, or other charts that indicate the patient's status. More sophisticated operations performed by the computer 69 include operating algorithms that process data from the SSMD 150 in various ways. For example, the algorithms may analyze trends in measurements made by the SSMD to predict values of future measurements before they are actually made. Or they may collectively analyze multiple parameters measured by the SSMD (e.g. both ACT and H&H) to predict a physiological state corresponding to the patient.

As a particular example, during a PCI procedure, the fully integrated measurement system 152 featuring the SSMD 150 is connected to a patient in the Cath Lab. The surgeon performing the procedure typically requires ACT values every 30 minutes or so; it is particularly important that measurements are made each time the surgeon makes certain interactions with the patient, e.g. places a balloon catheter or stent, as described above with reference to FIG. 1. With the SSMD 150, the surgeon can focus on the procedure at hand and simply view the touchpanel display 77 to get ACT values that are measurement periodically or, as described, on demand. In some embodiments, as described below with reference to FIGS. 20 and 21, ACT and H&H values are measured in a continuous manner using a predictive algorithm. Here, the numerical values representing these parameters are continuously streamed to the touchpanel display 77 for the surgeon to view.

During post-surgery recovery, the fully integrated measurement system 152 and SSMD 150 can potentially ameliorate life-threatening conditions such as sepsis and internal bleeds which are indicated by, respectively, lactate, lactic acid, and H&H values corresponding to the patient. Typically, these values are measured in the hospital's hematology laboratory, a process that can take hours; the SSMD 150 can make comparable measurements in a matter of minutes. For sepsis, lactate is a chemical naturally produced by the body to fuel the cells during times of stress. Its presence in elevated quantities is commonly associated with sepsis and severe inflammatory response syndrome. When blood is lost, the body quickly pulls water from tissues outside the bloodstream in an attempt to keep the blood vessels filled. As a result, the blood is diluted, and the hematocrit (the percentage of red blood cells in the total amount of blood in the body, or blood volume) is reduced. In these and other cases, the SSMD 150 can detect the appropriate parameter, forward it to the hospital MR, where a clinician can then view it to diagnose the patient.

FIGS. 5A-5F show more specifically how an SSMD 150 attached to an IV pole 153 performs the following: 1) aspirates blood from the patient with the blood-extraction component 99; 2) makes a measurement with a measurement cell 102 within the measurement component 100; 3) flushes the system with saline to remove any residual blood from the system; and 4) then repeats the process with a new measurement cell 102 within the measurement component 100. In these figures, the measurement cell 102 is part of a circular array 163 of cells (like that shown in FIG. 3), and also includes a waste cup 168 which stores blood and saline used to flush the lines after a measurement is complete.

Referring first to FIG. 5A, the SSMD 150 shown in this figure is about to start a measurement. Here, the blood-extraction component 99 features a syringe 98 coupled to a computer-controlled linear actuator 179. Prior to any blood extraction, a plunger within the syringe 98 is pushed fully forward. A first segment of flexible tube 181 connects the syringe 98 to a first computer-controlled solenoid valve 173, which in turn features a fluid path to a measurement head 177 that deposits blood, reagents, and saline (for flushing the system) into a measurement cell 102. The measurement cell 102 is incorporated into the circular array 163 of cells, which in turn is rotated with another computer-controlled solenoid valve (not shown in the figure). A second segment of flexible tube 159 connects a saline bag (not shown in the figure, but similar to component 154 in FIG. 4) which provides saline to a second computer-controlled solenoid valve 174. A third segment of flexible tube 104 connects to an in-dwelling catheter inserted in a patient (not shown in the figure, but similar to component 158 in FIG. 4).

FIG. 513 shows a measurement upon initiation. Computer code operating on the SSMD 150 activates the linear actuator 179, which in turn pulls back the plunger on the syringe 98. The computer code also moves both the first 173 and second 174 solenoid valves such that suction from the syringe 98 aspirates blood from the patient, through the third segment of flexible tube 104, and finally into the first segment of flexible tube 181, where it is held for a short period of time.

FIG. 5C shows how blood aspirated from the patient flows into a measurement cell 102. Once the blood-extraction system 99 deposits blood in the first segment of flexible tube 181, computer code again moves both the first 173 and second 174 solenoid valves. The computer code also moves the linear actuator 179 forward, thus depressing the plunger on the syringe 98 and pushing blood from the first segment of flexible tube 181 past the first solenoid valve 173, and into the measurement head 177, which delivers it to a measurement cell 102 within the circular array 163. For the measurement shown in the figure, no reagent is used, and clotting is instigated with a clotting agent, as described above with reference to FIG. 2, i.e. blood is delivered to the measurement cell 102, and the magnetic ball (not shown in the figure) is rapidly moved back and forth with the magnetically coupled linear actuation (also not shown in the figure) so that the clotting agent is mixed with the blood to activate clotting. Once the blood and clotting agent are mixed, measurement of ACT and H&H proceeds as described above. To measure something other than these parameters, reagents can be mixed with the blood sample using a comparable methodology.

FIG. 5D shows how saline is used to flush the segments of flexible tube in preparation for a follow-on measurement. Here, once again, computer code adjusts the first 173 and second 174 solenoid valve and the linear actuator 179, the latter of which pulls back the syringe's plunger to draw saline from an IV bag (again, similar to the IV bag 154 shown in FIG. 4, but not shown in this figure) through the second segment of flexible tube 159, second solenoid valve 174, first solenoid valve 173, and finally into the first segment of flexible tube 181 where, like the blood shown in FIG. 5B, it sits for a brief moment. FIG. 5E shows how, at this point, computer code moves the first solenoid valve 173 and linear actuator 179, which pushes the syringe's plunger a partial distance to drive saline from the first segment of flexible tube 181, past the first solenoid valve 173 and measurement head 177, and finally into the waste cup 168. This flushes all lines not connected to the patient of all residual blood. Then, as shown in FIG. 5F, computer code moves the first 173 and second 174 solenoid valves, and drives fully forward the syringe's plunger to push a volume of saline through the first 173 and second 174 solenoid valves, through the third segment of flexible tube 104 and back into the patient. This clears the remaining line (the third segment of flexible tube 104) of any residual fluids. As a final step, computer code rotates the circular array 163, as indicated by arrow 169a, to position a new measurement cell underneath the measurement head 177. A new measurement then commences, as described above.

In embodiments, the blood-extraction component 99 shown in FIGS. 5A-F connects to a manifold (e.g. component 85 in FIG. 1), which in turn connects to the patient's arterial system. Here, the blood that the blood-extraction component extracts is fully oxygenated arterial blood that the measurement component then tests as described above. Alternatively, the blood-extraction component couples to the patient's venous system, and extracts partially oxygenated venous blood that is then sent to the measurement component for testing. An assumption here is that the degree of oxygenation in the blood has little to no impact on its coagulation. In this embodiment, the blood-extraction component typically includes a venous catheter that is inserted into the patient's vein prior to measurement, e.g. before the PCI procedure. The venous catheter couples to a syringe (e.g. component 98 in FIG. 1) and computer-controlled linear actuator (component 179 in FIG. 1), which then draws a blood sample in a manner similar to that shown in FIGS. 5A-F. In related embodiments, such as that shown in FIGS. 6A-6C and described in detail below, the blood-extraction component includes other mechanisms for drawing blood, e.g. a computer-controlled retractable sheath that periodically inserts into the catheter and is disposed past valves in the vein so that it can better sample flowing blood. Here, the retractable sheath couples to the syringe, which extracts blood when moved by the linear actuator. The sheath is then removed once blood is drawn.

Periodically inserting and extracting the sheath through this mechanism avoids clotting near the sheath's opening, which can occur if it is left in the vein for too long. Additionally, clotting can be further reduced by inserting the sheath past a valve in the patient's venous system that is proximal to the insertion site of the catheter. Here, ‘fresh’ blood also flows at a relatively high rate, and is ideal for extracting prior to delivery to the measurement component.

Referring now to FIGS. 6A-6C, another embodiment of the blood-extraction system features a specialized catheter 180 inserted in a patient's vein 182. During measurements, the catheter 180 automatically draws blood and transfers it to the measurement component described above (FIG. 6A), and then between measurements withdraws (FIG. 6B) to avoid clotting and hemolysis near the catheter's tip 189. The catheter 180 features a flexible IV tube 186a, 186b, 186c which encloses a retractable sheath 185a, 185b, 185c. Middle portions of the flexible IV tube 186b and retractable sheath 185b connect to a front face of a manifold/fitting 190 that includes internal channels 198, 199 that effectively separate the coupled, co-centric portions of the flexible IV tube 186b and retractable sheath 185b. Once separated, distal portions 185c, 186c of these components disposed outside the vein 182 exit a back face of the manifold/fitting 190. A movable mount 191 attaches to a computer-controlled linear actuator (not shown in the figure) that translates back and forth, as indicated by arrow 194a, 194b. The distal, out-of-vein portion of the retractable sheath 185c passes through a relatively small opening 197 in the mount 191, which secures it so that both the retractable sheath 185c and movable mount 191 traverse in concert, as indicated by arrows 194a, 194b. Conversely, the distal, out-of-vein portion of the flexible IV tube 186c passes through a relatively large opening 196 in the mount 191 which does not secure it in any way, but instead slides along its outer surface when the computer-controlled linear actuator pulls the mount 191 away from the manifold/fitting 190; this means the flexible IV tube does not move in-between measurements. An end portion of the distal, out-of-vein portion of the flexible IV tube 186c ultimately connects to an infusion system (e.g. a pump) that delivers, e.g., saline for flushing the line and/or heparin to the patient, this is indicated by arrow 192. The mated distal, out-of-vein portion of the retractable sheath 185c connects directly to the measurement component, as indicated by arrow 193. With this configuration, the computer-controlled linear actuator can move the mount 191 away from the manifold/fitting 190 and retract all portions of the retractable sheath 185a, 185b, 185c, as indicated by arrow 194b, while keeping all portions of the flexible IV tube 186a, 186b, 186c in place within the vein 182.

To measure ACT, PTT, and other parameters from a patient's blood, a clinician inserts the specialized catheter 180 into the patient's vein 182, as shown in FIGS. 6A and 6B. The vein 182, for example, may be the patient's radial vein, or any other vein located on their body, and preferably located in an easily accessible location, e.g. their arm or hand. As shown in the figures, the catheter can be inserted so that its tip 189 is located between a first valve with open flaps 183a, 183b, and a second valve with closed flaps 184a, 184b. (Note: for simplicity, in both these figures the first valve is shown as temporarily open, and the second valve as temporarily closed; for an actual patient, these valves will open and close periodically to move blood within the vein.) During a measurement, as shown in FIG. 6A, the computer-controlled linear actuator pushes the movable mount 191 up against the manifold/fitting 190, thereby moving the portion of the retractable sheath 185a past the tip 189 of the distal portion of flexible IV tubing 186a and the open flaps 183a, 183b of the first valve. This action moves the distal portion of the retractable sheath 185a into a region where blood tends to flow a higher velocity, as indicated by arrow 187, as compared to blood near the tip 189 where blood flows at a relatively lower velocity, as indicated by arrow 188. The blood flowing at a relatively high velocity is freely circulating and lacks any small clots and/or cells impacted by hemolysis that tend to congregate around the tip 189; it is considered fresh and better for testing ACT, PTT, and other parameters. A pump (not shown in the figure) connected to the distal, out-of-the-vein portion of the retractable sheath 185c draws out a small volume of blood, typically between 0.1-1.0 ml over a period of a few seconds, which passes to the measurement component for testing, as indicated by arrow 193.

When the measurement is complete, the computer-controlled linear actuator pulls the mount 191 away from the manifold/fitting 190, thereby pulling the entire retractable sheath, and most importantly, its distal in-vein portion 185a past the open flaps 183a, 183b of the first valve, and past the tip 189, where it remains until a new measurement initiates. This effectively shields it from blood and, when coupled with heparin flowing through the distal portion of the flexible IV tube 186a, prevents small clots from blocking flow within the retractable sheath 185a, 185b, 185c.

The above-described process is then repeated for each new measurement.

In other embodiments related to FIGS. 6A-6C, the linear actuator that translates the retractable sheath can be replaced with other mechanisms. For example, a mechanism based on one or more rotating gears, e.g. ‘rollers’ that are rotated by a computer-controlled stepper motor, can include ‘teeth’ that grip the retractable sheath shown in these figures and pull it in and out of the flexible IV tube to achieve the same results as shown above. Other similar mechanical mechanisms can be used for this purpose.

### 4. Examples of POC Devices in the Prior Art

Because it is fully automated and makes measurements in a quasi-continuous manner, the SSMD, as described above, improves on measurements for coagulation parameters and blood-based biomarkers that are made using: 1) existing POC devices; and 2) manual assays. In addition to the convenience it offers, the SSMD's automated, multiplexed measurements of parameters like ACT, H&H, and other blood-based compounds allows for a closed-loop system to address and treat patients receiving heparin during surgery and post-surgery recovery. As well, a surgeon in the Cath Lab can receive real-time, critical information and focus on the highly technical procedure at hand, with fewer interruptions, improved procedural turn-around time that minimizes human error. This approach features additional benefits of reducing volumes of manual blood aspirations and removing the risk of air or blood clots entering the system that can be catastrophic. Multiplexing multiple measurements with a single device integrated into a closed-loop system provides real-time measurements of critical biomarkers that allows rapid response to derangements to maximize efficiency and remove human error and delays.

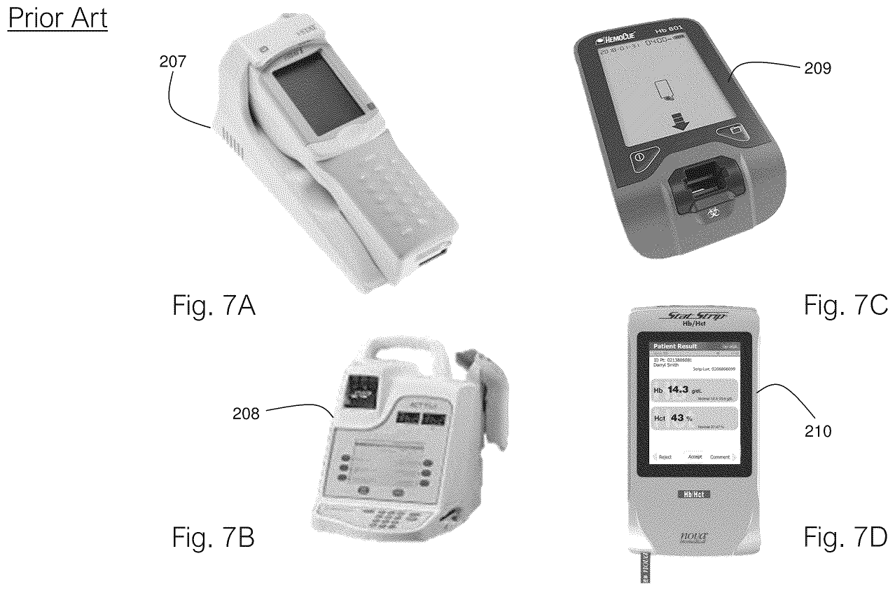

FIGS. 7A and 7B show two POC devices 207, 208 of the prior art that measure ACT. The first device 207, the i-STAT 1 (manufactured by Abbot), is a portable blood analyzer that measures ACT using an optical technique. The i-STAT 1 works when a clinician applies a few drops of blood onto a cartridge that is then inserted into the device. Results are available within the time of a conventional ACT (about 250 seconds) plus an additional 30 seconds for processing. The second device 208, the ACT Plus (manufactured by Medtronic), is a portable POC system that measures ACT levels using electrical resistance. Both the i-STAT 1 and ACT plus devices use kaolin (layered silicate mineral, similar to clay) as a reagent for blood clotting.

FIGS. 7C and 7D show two POC devices 209, 210 for measuring hemoglobin, a component of H&H (note: measuring hematocrit, as described above, requires the sample to be centrifuged to separate cellular and plasma components). The device 209 in FIG. 7C is the HemoCue (manufactured by Danaher), features a disposable cuvette that uses capillary action to take on blood. Sodium deoxycholate is then added to the cuvette to lyse the red blood cells, which releases hemoglobin. In the presence of added sodium nitrite, the released hemoglobin converts to methemoglobin. Methemoglobin, with sodium azide, yields azide methemoglobin; this sample, in turn, can be measured optically at two optical wavelengths: 1) 570 nm to determine methemoglobin; and 2) 880 nm to generate a signal that accounts for sample turbidity. The second device 210, the StatStrip (manufactured by Nova Biomedical), is a lightweight POC device that measures hemoglobin levels using capillary blood samples extracted from a patient using a fingerstick-inducing lancet. Following the fingerstick, the capillary blood is absorbed by a low-cost test strip, which then performs an optical testing similar to that used by the HemoCue device.

### 5. Measurement Systems Used in the SSMD

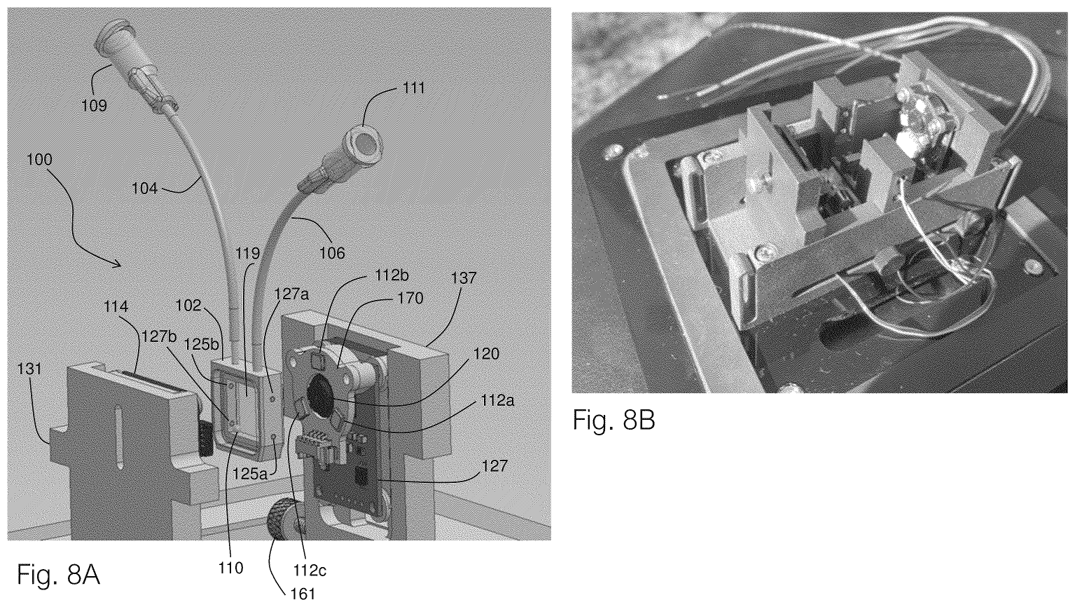

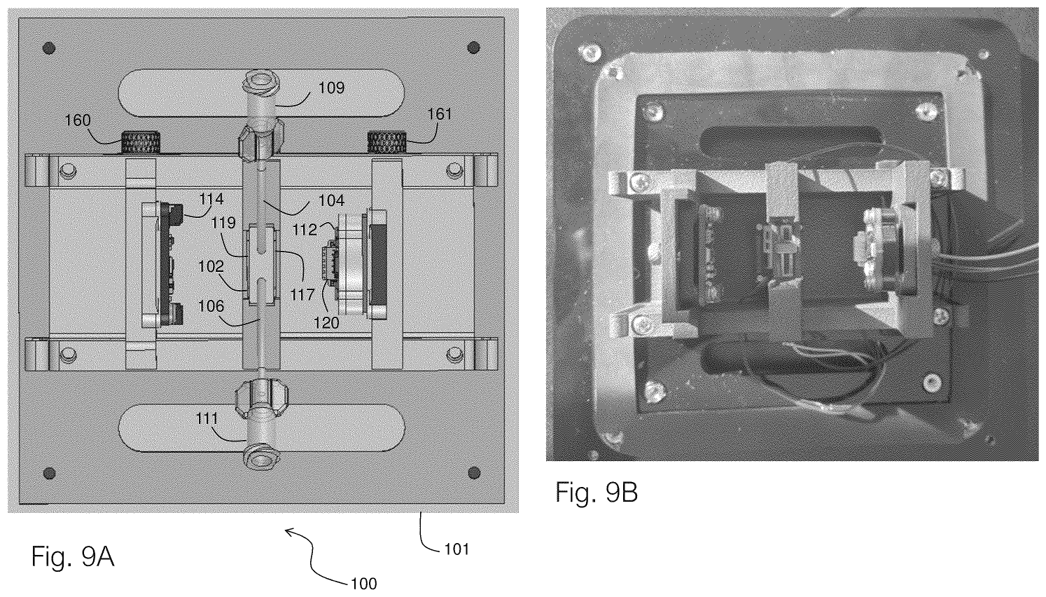

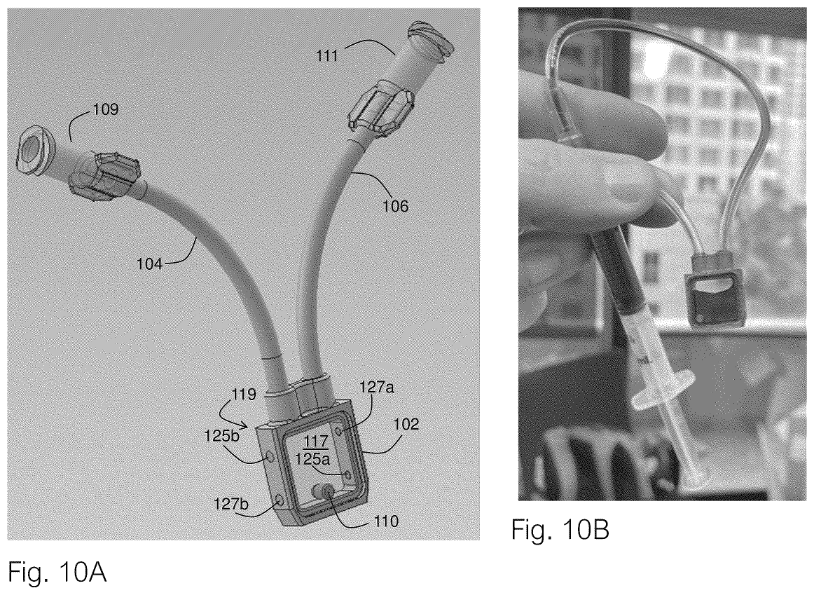

FIGS. 8A and 8B show a side view of, respectively, a mechanical drawing and photograph of the measurement cell 102 and measurement component 100 used within the SSMD. FIGS. 9A and 9B show a top view and photograph of, respectively, these same components. And FIGS. 10A and 10B show, respectively, a front view and photograph of the measurement cell 102. In the photograph shown in FIG. 10B, the measurement cell is filled with a liquid substance having the same viscosity and optical properties as human blood.

During a measurement, the measurement cell 102 receives blood through a first automated pipetting system 104, and reagents through a second automated pipetting system 106. The first 104 and second 106 automated pipetting systems terminate, respectively, with first 109 and second 111 leak-proof connectors that connect to the various segments of flexible tubing shown in FIGS. 5A-5F to receive blood and reagents.

The measurement cell 102 encloses a metal ball 110 within a plastic cell featuring optically transparent windows 117, 119 on both faces. The sides of the measurement cell, near its top and bottom portions, features pressed-in metal pins 125a, 125b, 127a, 127b functioning as sense (125a, 125b) and drive (127a, 127b) electrodes for the electrical impedance/reactance measurements described above. During a measurement, the measurement cell 102 is slid into a mated slot (not shown in the figure), typically in an automated manner, that includes spring-loaded metal contacts (e.g. pogo pins) that press against the metal pins 125a, 125b, 127a, 127b and connect them to appropriate sense/drive terminals in an underlying circuit.

Alternatively, in embodiments, the transparent windows 117, 119 are coated with ITO electrodes.

To measure ACT, a magnet attached to a computer-controlled linear actuator (not shown in the figure, but described in more detail with reference to FIG. 11A) moves back and forth underneath the measurement cell, causing the metal ball 110 to move in a commensurate manner. Clotting blood gradually impedes movement of the metal ball 110, eventually causing it to cease completely. A small-scale video camera 120 records time-dependent images indicating movement of the metal ball 110 that can be analyzed with an algorithm, as described above, to determine ACT. The small-scale video camera 120 is controlled by a circuit board 127 that includes a microprocessor, image-processing electronics, and other electronics for power management and other components. A first mounting component 137 supports both the circuit board 127 and small-scale video camera 120.

FIG. 11B shows a photograph of the measurement cell 102 with the enclosed metal ball 110. The photograph was taken with the small-scale video camera 120 shown in more detail in FIGS. 9A and 9B. Here, the measurement cell 102 is not filled with blood, and thus magnetically controlled motion of the metal ball is unimpeded. During an actual measurement, an image-processing software system, such as one based on ML/AI, would analyze multiple photographs liked that shown in FIGS. 11B, all taken at different points in time, to track the metal ball within the blood-filled cell. As blood within the measurement cell 102 clots, lateral movement of the metal ball will be impeded. The ball's exact motion is determined by extracting a two-dimensional, time-dependent plot, similar to those shown in FIGS. 12A-D, with the image-processing software system. Analysis of these plots yields ACT, as described in more detail below.

To make an optical spectroscopic measurement, similar to that shown in FIGS. 18A and 19, a circuit board 170 shaped like an annular ring supports three white-light LEDs 112a, 112b, 112c. The white-light LEDs 112a, 112b, 112c emit radiation ranging from about λ=380-1000 nm that fully illuminates the measurement cell. The circuit board 170 is placed on top of the small-scale video camera 120 so that it surrounds a lens used therein. During a measurement, the circuit board 127 supplies power to the three white-light LEDs 112a, 112b, 112c, causing them to emit optical radiation in the spectral regions described above. The radiation passes through the optically transparent windows 117, 119 of the measurement cell 102, where it is partially absorbed and scattered by the blood sample therein. The amount of optical absorption and scattering depends on the degree of clotting in the blood, and additionally any chemical reaction that may occur in the cell 102, e.g. due to an added reagent, such as an aptamer or aptamer/fluorophore compound. In these applications, a ‘baseline measurement’ is typically made before any reagent is added to the blood sample.

Radiation that passes through the measurement cell and the blood within is then sensed with a photodetector 114 mounted on a separate circuit board (not shown in the figure), which is turn is supported by a second mounting component 131. Prior making an optical spectroscopic measurement of blood, the measurement component 100 first makes a baseline measurement wherein the above-described optical system measure a blood sample without any reagent (or alternatively an empty measurement cell). This entails powering on the three white-light LEDs 112a, 112b, 112c with the circuit board 127 and detecting radiation with the photodetector. Once the baseline measurement is complete, the automated pipetting system 104 loads blood into the measurement cell, where it first mixes with the clotting agent. The circuit board 127 then powers on the white-light LEDs 112a, 112b, 112c, which generate broadband optical radiation that passes through the measurement cell and is detected by the photodetector 114. As described above, the front face of the photodetector 114 features computer-controlled optical filters that pass discrete, specific bands of radiation, each associated with optical wavelengths as indicated by the red triangles in FIG. 19.

A series of setscrews 160, 161 allow the first 137 and second 131 mounting components to be laterally adjusted during manufacturing to improve collection of signals used for the imaging and optical spectroscopic measurements. In alternate embodiments, these setscrews 160, 161 are connected to computer-controlled actuators that move them in an automated manner. A secure mounting plate 101 holds all the above-mentioned components in place.

Referring to FIG. 11A, different approaches based on magnets may be used to move the metal ball 110 within the measurement cell 102, as indicated in the figure by arrows 147. A preferred approach, as described in detail above, is laterally translating a magnet 116 disposed underneath the cell 102 (e.g. using a linear actuator), as indicated by arrows 118a, 118b. This method has the advantage that such lateral motion is relatively easy to control using a small microcontroller. In embodiments, for example, the speed and span of the lateral motion can be controlled using computer code that enacts pulse wave modulation (herein “PWM”) to control a small motor within the linear actuator. The disadvantage of this approach is that it involves a physically moving component, and these, in turn, have a relatively high rate of failure, especially when deployed over extended time periods.

FIG. 11A also shows an alternative magnetic approach to laterally moving the metal ball 110 within the measurement cell 102. Here, two stationary electromagnets 141, 143 are positioned proximal to distal ends of the cell 102. The magnets 141, 143 generate a magnetic field when a voltage (e.g. a potential difference of 5V, as shown in the figure) is applied. Thus, by using a computer-controlled power-management system, voltage can be alternatively applied to the first electromagnet 141 for a period of time (e.g. a ‘dwell time’ of 1-2 seconds), and then switched to the second electromagnet 143 for a similar dwell time. A computer-controlled system like this that alternates the applied voltage back-and-forth moves the metal ball 110 according to the arrow 147 in a manner that's similar to motion driven with a moving magnet 116 and linear actuator. This approach has several advantages over that described previously, mainly that: 1) it does not include and moving parts; and 2) it potentially provides more control (e.g. by increasing the applied voltage or dwell time) over the movement.

FIGS. 12A-12D show simulated time-domain signals calculated with a mathematical model that represents how the metal ball, as described above with reference to FIG. 11A, is driven by the linear actuator and magnet to move within the measurement cell as blood that surrounds it clots. FIGS. 12E-12F show frequency-domain signals calculated by taking a Fourier Transform of the time-domain data in FIG. 12A-12D.

Referring first to FIG. 12A, as fresh blood is first introduced to the measurement cell, it is completely unclotted and offers little resistance to the magnetically driven metal ball. Both this signal and its Fourier Transform in FIG. 12E indicate that the blood is completely unclotted. Specifically, the time-domain signal in FIG. 12A is undamped, and resembles the response from a frictionless harmonic oscillator; the corresponding frequency-domain signal in FIG. 12E features a sharp (technically infinitely narrow) peak 140a with a frequency indicated by the dashed line 142. The half-width of the peak 142, when fit with a Gaussian function, is γ=0 Hz as indicated in FIG. 12E.

As blood begins to clot, it provides more resistance to the metal ball, and signals from the previously undamped harmonic oscillator indicated in FIGS. 12A and 12E begin to dampen. FIGS. 12B and 12F, for example, taken from moderately clotted blood, indicate an exponential decay in the time-domain signal; the Fourier Transform in FIG. 12F thus shows a peak 140b at the same frequency as that in FIG. 12E (as indicated by the dashed line 142) with a measurable half-width of γ=0.5 Hz. Note that γ determined from the Fourier Transform is the time constant in the exponential function describing the decay. As clotting significantly increases, as shown in FIGS. 12C and 12G, damping increases in a commensurate manner, causing the time-domain signal to decay in a more rapid manner, thereby increasing the width of the peak 140c in the frequency-domain signal to γ=2.0 Hz. At a time consistent with ACT, as indicated by FIGS. 12D and 12H, blood is completely clotted in the measurement cell, and the time-domain signal is fully overdamped and shows no oscillations whatsoever. The frequency-domain signal, in turn, shows no measurable peak (the dashed line 142 indicates where the peak would normally be located if the signal was not completely overdamped; as indicated in FIG. 12H, the damping for the time-domain signal in FIG. 11D was γ=15.0 Hz).

### 6. Algorithms

As described above, the SSMD provides three unique approaches for measuring ACT: 1) time-resolved optical absorption; 2) time-resolved imaging of a mechanical event (a moving metal ball controlled by stationary or moving magnets); and 3) time-resolved electrical impedance or reactance spectroscopy, using either a single frequency or multiple frequencies of injected electrical current. Additionally, simply capturing an image of clotting blood within the sample cell with the video camera described above, and then analyzing these results with image processing and an associated algorithm, may yield ACT. This is because the physical structure of blood changes as it clots over time, and this is likely manifested as a change in the associated image of the sample cell.

The multiple measurements of ACT made by the SSMD may be collectively processed in a variety of ways to yield a single, cumulative measurement. For example, they can be averaged together, with any ACT value falling outside a pre-determined region (e.g. more than 1 standard deviation from the mean ACT value) being discarded. Or the average or weighted average can be calculated, with ACT values determined from time-dependent signals that fail to meet pre-determined thresholds for noise discarded from the average. This could occur, for example, if blood within the measurement cell coagulated in a particularly non-uniform manner, thereby indicating the measurement technique used to determine ACT in this instance would be flawed. ACT is known to be somewhat device-specific (e.g. the prior art devices shown in FIGS. 7A and 7B may yield different values according to the algorithms used therein). Thus, future clinical studies may indicate that one approach used by the SSMD (e.g. time-resolved optical absorption, as indicated in FIGS. 18A and 19) may correlate better with a particular device (e.g. the i-STAT 1, manufactured by Abbot), while another (e.g. time-resolved impedance, as indicated in FIGS. 15A, 15B, and 27D) may correlate better with another device (e.g. the ACT Plus, manufactured by Medtronic). Here, the SSMD could report multiple ACT values, e.g. an ‘Abbot value’ that is more consistent with Abbot's measurement, and a ‘Medtronic value’ that is more consistent with Medtronic's value. In general, the SSMD is designed to report multiple ACT values.

In other embodiments, PTT may be calculated alongside of ACT, and these two parameters are then processed algorithmically together. As described above, PTT is widely considered to be a more accurate metric for coagulation, but is rarely used in the Cath Lab because of the time-consuming and inconvenient nature of the test used to determine it. In embodiments, PTT may be determined initially for a patient; ACT is then determined independently using each of the three techniques described herein (optical, impedance, mechanical/imaging). The ACT test that best agrees with the PTT measurements is then used going forward.

In all cases, the microprocessor controlling the SSMD may run computer code that utilizes different signal-processing techniques to analyze signals measured by the SSMD's various sensors to extract ACT values. In embodiments, for example, the computer code may run numerical ‘fitting’ algorithms to analyze time-dependent waveforms, such as the optically generated waveform in FIG. 17B, or the impedance-generated waveform in FIG. 27D, to extract the ACT values. For the fitting algorithm, a pre-determined mathematical function is derived from a physiological process that describes the blood clotting, e.g. a linear function coupled with an exponential function. The mathematical function is then input into computer code that runs the fitting algorithm, which then iteratively varies parameters associated with the function (e.g. an exponential time constant associated with a non-linear function; a slope and y-intercept associated with a linear function) until the pre-determined mathematical function best matches the time-dependent waveform measured by the SSMD. The computer code then calculates ACT values from the ‘fit’ and its associated parameters.

In other embodiments, in place of the fitting algorithm, the computer code used by the SSMD analyzes time-dependent waveforms to determine changes that, in turn, indicate ACT. For example, referring to the impedance-generated waveform in FIG. 27D, the rapid change in value near 450 seconds indicates ACT; computer code used by the SSMD can detect this by comparing the signal to pre-determined threshold values, or by taking a mathematical derivative of the signal and then analyzing the derivative for the largest rate of change.

For images collected by the small-scale camera, the computer code may deploy image-processing or pattern-recognition algorithms that compare images collected by the SSMD to pre-determined images that indicate clotting blood. Likewise, the SSMD can use the above-described fitting approach to analyze time-domain signals extracted from images collected with this approach; FIGS. 12A-12D show simulated examples of such signals. More specifically, and as described above, the time-domain signals shown in these figures can be modeled by the motion of a damped harmonic oscillator. A fitting function that represents this system features exponential damping component (e.g. e^{-γ}, where γ is a damping constant and t is time) multiplied by a sine wave (e.g. sine(ωt)), where t is again time and ω is a frequency corresponding to the oscillatory nature of the metal ball driven by the magnet within the measurement cell. Here, ω is determined by the periodic movement of the linear actuator and magnet, and thus is a known quantity; γ indicates clotting time and, thus, ACT. Thus, an algorithm that first measures a time-domain signal, then calculates its Fourier Transform, and finally determines a peak in this frequency-domain signal (e.g. components 140a, 140b, 140c in, respectively, FIGS. 12E-12G) can used to determine ACT. In embodiments, damping measured before blood is fully clotted (e.g. signals from the moderately clotted blood, as shown in FIGS. 12B and 12F) can be analyzed with algorithms that predict ACT (and other parameters). Such algorithms, for example, may rely on AI and ML. In similar examples, the algorithms may be based on relatively simple look-up tables determined beforehand, e.g. from clinical studies conducted with the SSMD and a gold-standard reference to determine the absolute value of ACT.

For H&H, and particularly hemoglobin, the computer code may deploy peak-detection algorithms in the frequency domain to determine the parameter of interest. For example, as indicated by FIG. 18A, a well-defined peak appears in an absorption spectrum of whole blood at around λ=680 nm. This peak gets less pronounced as the whole blood sample gets diluted (using deionized water, the percentage of this compound ranged from 66.7% to 99.3%, as indicated in the figure). FIG. 18B shows the peak amplitude of signal at λ=680 nm as a function of dilution. As shown in this scatter plot, the amplitude decreases in a non-linear manner as dilution is increased.

In embodiments, to determine hemoglobin concentration in the blood, the peak-detection algorithm determines an amplitude corresponding to the signal measured at a predetermined wavelength (e.g. λ=680 nm) with the SSMD, and compares it to a pre-determined ‘look-up table’ to estimate the concentration. The look-up table is typically determined beforehand using clinical studies that systematically vary the concentration of hemoglobin in whole blood samples, and then measure these samples with an optical spectrometer similar to that used in the SSMD and a gold-standard reference.

For measurements that require adding a reagent (e.g. an aptamer that binds to a specific protein) to the whole blood sample, and then measuring the result sample (typically using an optical technique), the algorithm typically involves analyzing pre-determined, well-defined peaks corresponding to the aptamer-protein compound, or an optically active compound bound to the aptamer, and then analyzing the resultant absorption spectrum as described above. In some cases, the optically active compound involved in this chemical reaction exhibits such a profound colorimetric change that the small-scale camera can detect it by detecting an image and using image-processing or pattern-recognition algorithms. Here, the image is processed with the algorithm, and the results of this analysis are compared to a look-up table featuring known concentrations of the sample to determine the concentration of the aptamer-bound protein. Other types of algorithms may also be used according to the invention.

The optical, impedance, and image-processing algorithms described herein make episodic measurements of ACT, H&H, and other blood-based parameters. The frequency at which these measurements can occur is ultimately limited by the time required to complete the measurement. For example, a ‘therapeutic’ value of ACT is approximately 250 seconds, so if the SSMD only includes a single measurement component, then ACT measures cannot occur at a frequency greater than about 1 measurement every 250 seconds.

To increase this measurement frequency, the SSMD may use an array of measurement cells, such as the circular or linear arrays shown in FIGS. 2B and 2C, respectively. Here, for example, at time t=0 a measurement is made with a first cell; this takes approximately 250 seconds. While the SSMD calculates the first ACT value, the linear array shifts to a new cell after a time period less than the ACT measurement time (e.g. t=60 seconds), and the SSMD makes a second measurement, with its results also taking about 250 seconds to generate. This approach effectively increases the frequency that ACT measurements can be made.

In other embodiments, the microprocessor running on the SSMD may apply algorithms to the parameters episodically measured by the measurement component to ‘estimate’ future values of these parameters, and by doing so may provide quasi-continuous measurements that may benefit the clinician. FIGS. 20 and 21 show flow charts representing algorithms for measuring, respectively, ACT and H&H using this approach. More specifically, the figures show schematic drawings of algorithms operating on computer code running on the microprocessor within the SSMD to measure ACT and H&H. Referring first to FIG. 20, the algorithm for the ACT measurement (box 211) receives an episodic measurement value from the measurement component within the SSMD (as indicated by box 212) which indicates an initial value for ACT at time t=0 (ACTₜ₌₀). this value represents an absolute value of ACT, and, as described above, is made with the SSMD at a maximum frequency corresponding to the time it takes to complete a conventional measurement (e.g. about 250 seconds). Once this is complete, continuous measurements (as indicated by the box 213) can be made at a relatively high frequency, e.g. once every second. For example, the continuous measurement may look at features of signals that can be measured rapidly by the measurement component—e.g. optical and impedance signals, as opposed to images of the moving metal ball with the video camera—that contain features that relate to changes in ACT. Signal components that may indicate this include slight changes in optical absorption or trends in the time-dependent impedance signal. These signal components indicate a time-dependent change in ACT at time t=x, measured as described above to yield a value of AACTₜ₌ₓ. Such an algorithm, for example, may be based on a linear regression algorithm or polynomial expansion and determined during a clinical trial using a large number of blood samples processed with varying levels of heparin and measured with both electrical impedance and conventional POC devices that determine ACT. A fitting algorithm, similar to that described above, may also be used here to analyze the time-dependent waveforms. Ultimately such an approach yields continuous, time-dependent values of ACT that can be validated by making periodic measurements with a POC device, as shown by the graph in FIG. 22.

Referring to FIG. 21, a similar approach can be used for H&H measurements. Here, a general algorithm (indicated by box 221) receives initial, episodic values for H&H at time t=0 (H&Hₜ₌₀), as determined by the measurement component as indicated by box 222. These values can typically be determined much faster than ACT. Once this is determined, the processing system calculates higher-frequency, time-dependent measurement inputs from the measurement component (indicated by box 223) that yield H&H values at time t=x (ΔH&Hₜ₌ₓ).

Similar approaches can be used for blood-based parameters other than ACT, PTT, and H&H, as described in more detail above.

### 7. Other Embodiments

Other embodiments are within the scope of the invention. For example, the SSMD can be coupled to a physiological monitoring device, such as a wearable device, to better characterize a patient during a procedure or post-surgery recovery. In this embodiment, clinical data characterizing the patient's vital signs and hemodynamic parameters, as measured with the monitoring device, can be combined with clinical data characterizing the patient's blood (e.g. ACT, hemoglobin) to predict patient decompensation.

In embodiments, for example, clinical data from both sources can be used to predict sepsis during post-surgery recovery. Markers for sepsis-related decompensation include increased lactic acid; this can be monitored using the SSMD and a reagent containing an aptamer that specifically binds to this biomarker. Examples of such aptamers that bind to lactic acid include those described in the following references, the contents of which are incorporated herein by reference: 1) Frith et al., ‘Towards development of aptamers that specifically bind to lactate dehydrogenase of Plasmodium falciparum through epitopic targeting’, Malar J. 2018; 17: 191 (published online May 3, 2018 doi: 10.1186/s12936-018-2336-z); and 2) Minagawa et al., ‘Modified DNA Aptamers for C-Reactive Protein and Lactate Dehydrogenase-5 with Sub-Nanomolar Affinities’, (published online Apr. 13, 2020, doi: 10.3390/ijms21082683). When coupled to a molecule that can be detected with one of the approaches described above, e.g. an assay using the small-scale camera to detect a colorimetric change, these aptameric compounds, or comparable derivatives thereof, can detect the concentration of lactic acid in the patient's blood. This value can then be collectively processed with physiological signals-such as an increase in heartrate or blood pressure, or a decrease in stroke volume—to determine a level of decompensation in the septic patient.

In a related embodiment, aptamers used to detect BNP can be combined with physiological signals in a manner similar to that described above to determine a patient's progressing towards congestive heart failure (herein “CHF”). Examples of such aptamers that bind to BNP include those described in the following references, the contents of which are incorporated herein by reference: 1) Grabowska et al. ‘Electrochemical Aptamer-Based Biosensors for the Detection of Cardiac Biomarkers’, ACS Omega 2018, 3, 12010-12018 (published online Sep. 26, 2018, doi: 10.1021/acsomega.8b01558); and 2) Bruno et al., ‘Preliminary Development of a DNA Aptamer-Magnetic Bead Capture Electrochemiluminescence Sandwich Assay for Brain Natriuretic Peptide’, Microchem J. 2014 Jul. 1; 115: 32-38 (published online Jul. 1, 2015, doi:10.1016/j.microc.2014.02.003).

The contents of each of these documents are incorporated herein by reference.

An increase in BNP indicates a patient's progression towards CHF. Likewise, this condition is typically associated with a decrease in the patient's stroke volume, and increase in heart rate, and an increase in respiration rate. As with sepsis, levels of BNP in the patient's blood, as determined with the SSMD, can be combined with changes in these physiological signals to determine the patient's progression towards CHF.

In embodiments, analytical models based on AI and/or ML can collectively process SSMD-determined proteins in the patient's blood with their physiological signals to determine the patient's disease state.

In embodiments, blood and reagents can be mixed (e.g. prior to being loaded in the measurement cell, or in the actual measurement cell) with a mechanical mixing component. This component can be, for example, a propeller-shaped device or something similar coupled to a rotary motor. Such a system would replace the magnetically controlled metal ball as used for mixing purposes, as described above. Here, because the metal ball is not present, both the optical and impedance-based approaches described above are used to measure ACT or PTT; the mechanical measurement featuring the camera is typically not used. Alternatively, the camera can collect time-dependent images of the propeller-shaped device while it is rotating, analyze these to determine a two-dimensional time-dependent signal, and then process this signal with the algorithms described above to determine ACT or PTT.

FIGS. 23A and 23B show a wearable device 310 that can be used to measure the above-mentioned physiological signals, which are then used in concert with the SSMD, as described above. The wearable device 310, which is worn on the chest of a patient 312, features first 330 and second 336 electronics components that connect through a cable 334. As shown in FIG. 25, the device 310 communicates with an external gateway, such as a touchpanel display 77, using an internal Bluetooth® or Wi-Fi transmitter (not shown in the figure). The first component 330 includes a sense 341, 347 and drive 342, 348 electrode leads that, during use, connect to a pair of disposable electrodes (e.g. Red Dot™ electrodes from 3M Inc.) to make a measurement. To measure bioelectric signals, the disposable electrodes typically include hydrogel and adhesive components that secure to a patient's skin, and an Ag/AgCl-coated plastic rivet that connects to the hydrogel and snaps into the electrode leads 341, 342. These electrodes are paired with a second sense 347 and drive 348 electrode leads within the second component 360. The sense electrode leads, when attached to the disposable electrodes, measure bio-electric signals from the patient 312 that are received by an electronic circuit within the first component 310 that includes a differential amplifier and amplifier chain configured to measure a time-dependent ECG signal, such as that shown in top traces of FIGS. 24A and 24B. The ECG signal typically include heartbeat induced pulses or ‘QRS complexes’, best shown as the pulsatile components in FIG. 24B, that software called ‘beatpicking software’ operating on a microcontroller within the device 310 analyzes to determine heart rate. Standard algorithms for beatpicking software (e.g. the Pan-Thompkins algorithm) are well-known in the art.

The drive electrode leads 342, 348 also connect to disposable electrodes during a measurement, and inject high-frequency, low-amperage current into the patient 312 to make bio-impedance and/or bio-reactance measurements, similar to those described above with reference to the SSMD. The bio-impedance and/or bio-reactance measurements yield a time-dependent waveform, similar to that shown in the bottom traces of FIGS. 24A and 24B, that when processed with software operating on a microcontroller within the device 310 yields pulses corresponding to cardiac and respiratory functions. As shown in these figures, during normal breathing and fast breathing, the time-dependent waveform features signal components corresponding to both respiratory (relatively low-frequency pulses) and cardiac (high-frequency pulses) behavior of the patient. When the patient holds their breath, as indicated by box 355 in FIG. 24, only the relatively high-frequency pulses corresponding to cardiac behavior are evident.

Algorithms for analyzing the bio-impedance and bio-reactance signals to determine respiratory rate and stroke volume are described, for example, in the following patent applications, the contents of which are incorporated herein by reference: U.S. Pat. No. 10,722,131, entitled ‘Body-worn system for continuous, noninvasive measurement of cardiac output, stroke volume, cardiac power, and blood pressure’ and U.S. Pat. No. 9,211,073, entitled ‘Necklace-shaped physiological monitor’.

The wearable device 310 can also include sensors that measure other physiological signals. For example, in the embodiment shown in FIGS. 23A and 23B, the first component 330 includes a microphone 332 that measures heart sounds from the patient, e.g. S1 and S2 heart sounds corresponding, respectively, to the closing of the mitral and tricuspid valves (S1) and aortic and pulmonary values (S2). In embodiments, the second component includes a reflective optical system 360 that includes a two-color LED 353 (emitting wavelengths near λ=660 and λ=900 nm) surrounded by an array 354 of six optical photodetectors. During a measurement, the array 354 detects optical radiation from the LED 353 at both wavelengths after it reflects off underlying capillaries to determine PPG waveforms corresponding thereto. Algorithms operating via computer code running on the wearable device's internal processor can process PPG waveforms to yield high-frequency AC and baseline, DC signal components at both wavelengths. The computer code can further process the AC and DC signal components to yield values of SpO₂. Such algorithms are described, for example, in the following patent applications, the contents of which are incorporated herein by reference: U.S. Pat. No. 10,813,562, entitled ‘Body-worn pulse oximeter’ and U.S. Pat. No. 9,211,073, entitled ‘Necklace-shaped physiological monitor’.

In other embodiments, the wearable device can feature form factors other than a patch. For example, it may be in the shape of a finger-worn ring.

FIG. 25 indicates how the SSMD 150 and wearable device 310 can simultaneously measure a patient 312 according to the invention. The SSMD 150 connects to the patient in a manner similar to that described with respect to FIG. 4, e.g. using an in-dwelling catheter 21. Using the blood-extraction and measurement components described above, it measures the presence and concentrations of blood-based compounds in the patient using the approach described above, and wirelessly transmits information describing this to the touchpanel display 77, as indicated by arrow 79. Simultaneously, the wearable device 310 measures physiological waveforms, which are then processed to determine vital signs and hemodynamic parameters as described above. The wearable device wirelessly transmits this information to the touchpanel display 77, as indicated by arrow 78. Algorithms operating on the touchpanel display, e.g. AI and ML algorithms, collectively process this information to determine the patient's physiological state.

In other embodiments, the measurement and blood-extraction components within the SSMD can feature other configurations. For example, as shown in FIGS. 26A, 26B, and 27A-27D, the SSMD can take the form of a small-scale, wearable patch 350. In embodiments, for example, the wearable SSMD patch 350 is embedded within the wearable device 310 of FIG. 23A. Like the SSMD described herein, the wearable SSMD patch 350 features a blood-extraction component 399 and a measurement component 300. The blood-extraction component 399 connects to a small-scale catheter 323 (e.g. a needle or filament) that, during use, presses into the patient's skin an extracts a small volume of blood or interstitial fluid. The catheter 323 connects to a microfluidics channel 310b that is formed from an underlying printed circuit board (herein “PCB”) substrate 306 attached to an overlying plastic cover 302 by an adhesive layer 304. The adhesive layer 304 includes a circular opening 329 so that, when the PCB substrate 306 and plastic cover 302 attach, the resultant structure forms a small ‘well’ 351, fed by the microfluidics channel 310a, 310b, for blood to pool, clot over time, and be subsequently measured. For this to happen, the plastic cover includes an opening 308 that facilitates the draw of blood through the microfluidics channel 310a, 310b and into the well 351 via capillary action. A top cover 301 forms a barrier above the plastic cover 302 to prevent oxygen from exposing the blood within the well 351.

The well 351 includes sense 312a, 312b and drive 317a, 317b electrodes that, once connected to an internal impedance circuit, make a measurement as described above. During this measurement, the drive electrodes 317a, 317b, which are located distally, inject high-frequency, low-amperage current into the well 351, which in turn samples the clotting blood as described above. The sense electrodes 310a, 310b, which are located internally, measure the electrical impedance encountered by the injected current in the clotting blood. FIGS. 27A-27D indicate this process in more detail. As shown by the image in FIG. 27A and plot in FIG. 27D, for this measurement, the impedance starts out at a relatively high level as blood enters the well. At t=450 s, as shown by the image in FIG. 27B, the blood begins to clot and electrical impedance measured by the measurement component 300 begins to drop rapidly, indicating that the blood's conductivity in the well 351 increases during coagulation. Eventually at around t=750 s the blood fully clots, as indicated by the image in FIG. 27C, and the impedance stays relatively constant. The time-dependent graph in FIG. 27D indicates the temporal evolution of this coagulation process. During a measurement, a microprocessor within SSMD patch 350 processes signals like this to determine ACT.

In embodiments, components used in the SSMD patch 350 shown in FIGS. 26A and 26B can be incorporated into form factors that are more easily worn on the patient's body, e.g. a form factor resembling a watch, ring, or bracelet.

In other embodiments, the SSMD patch 350 includes a transmissive or reflective optical system, similar to that shown in FIG. 2A but on a smaller, integrated scale. This embodiment of the optical system measures absorption spectra from blood within the well, which can then be processed as described above to determine H&H.

In other embodiments, as indicated by the flow chart in FIG. 28, the SSMD can be used in concert with an infusion pump to deliver heparin to a patient in a closed-loop process 369. Here, the SSMD's blood-removal component can feature two valves, and integrates with a heparin-delivery system, e.g. one that includes heparin (e.g. dissolved in a saline bag) an infusion pump, and a third value.

The closed-loop process 369 starts as the SSMD begins to acquire blood from the patient (step 370). To do this, its processing system opens a first valve in-line with a catheter disposed in the patient's arterial or, more preferably, venous system (step 371), and closes a second valve in-line with the measurement component and the measurement cell therein (step 372). The processing system then controls an actuator to deploy a syringe (step 373), which in turn extracts blood from the patient and temporarily stores it (step 374), e.g. in tubing in contact with the catheter.

Once the blood is stored (step 374), the processing system begins to load it into the measurement cell (step 375) by closing the first valve in-line with the patient's arterial/venous system (step 376) and opening the second valve in-line with the measurement component (step 377). The processing system then controls the same actuator used in step 373 to push the blood sample into the measurement cell (step 379) within the SSMD's measurement component.

The measurement component, using one (or all) of the above-described optical, impedance, and mechanical sensors coupled to the measurement cell, measures blood within the measurement cell to determine a corresponding ACT/PTT value (step 380). Based on this value, the processing system opens a third valve, separate from the blood-extraction component, that is in-line with an external infusion pump (step 381). The infusion pump may couple to the patient using the same catheter used in the blood-extraction component, or with a separate catheter. An algorithm running on the processing system uses the ACT/PTT value to first determine if the patient needs heparin, and if so how much (step 382). Here, the algorithm can be a simple look-up table incorporating pre-determined values of ACT/PTT values and corresponding heparin doses; the pre-determined values, for example, can be determined using clinical studies, EMR records, and values reported in the medical literature. In related embodiments, the algorithm additionally considers biometric data from the patient (e.g. their weight, age, gender, medical condition, typically determined from the EMR) to determine the heparin dose. Once it is determined, the processing system communicates the heparin dose to the infusion pump (step 383), e.g. through a wired or wireless interface, which then delivers it to the patient (step 384).

The closed-loop process 369 can be deployed with systems not worn on the patient (e.g. similar to that shown in FIGS. 4 and/or 25), or with a completely wearable system (e.g. similar to that shown in FIGS. 26A and 26B).

Still other embodiments are within scope of the invention. For example, the system described above can also include additional sensors for measuring other properties for human blood. The system can also include a three-axis digital accelerometer and a temperature sensor to measure from the patient, respectively, three time-dependent motion waveforms (along x, y, and z-axes) and temperature values within the SSMD. The optical sensor can include a heating element featuring a thin Kapton® film with embedded electrical conductors arranged in a serpentine pattern to increase perfusion and, subsequently, the strength optical signals.

In other embodiments, the system described above can be used for other surgical applications, or to measure ACT and H&H remotely from a patient's home. In this latter case, the system may be coupled to a cloud-based system that may also include AI and ML algorithms to process information.

In other embodiments, the entire SSMD may be heated (e.g. to 98.6°) to better match the temperature of the human body.

In other embodiments, the SSMD can include other systems that make additional measurements of the patient's blood. For example, the SSMD may include an additional sensor (e.g. one based on a microchannel plate incorporating one or more electrodes) that measures the pH of the blood sample. To make such a measurement, this new calibration preferably includes a PCO2 electrode, which is a conventional pH electrode (e.g. a glass, ion-selective electrode made of a doped glass membrane that is sensitive to hydrogen ions) surrounded by a layer of electrolyte solution and calibrated for PCO2. With this electrode, the actual pH of the blood and the partial pressure of CO2 in the blood (PaCO2) can be measured.

The reagent used for the ACT measurement is typically celite or kaolin, which activates a clotting reaction. Kaolin activates the intrinsic clotting pathway, thereby leading to the activation of a thrombin substrate that is not fibrinogen. In other embodiments, the SSMD includes an electrochemical sensor that indicates when the activation of a thrombin substrate is complete. The substrate used in this assay (H-D-phenylanyl-pipecolyl-arginine-p-amino-p-methoxydiphenylamine) mimics the thrombin-cleaved amide linkage found in fibrinogen. The product of the reaction consists of an electrochemically inert tripeptide (phenylalanyl-pipecolyl-arginine) and an electroactive compound (NH₃⁺—C₆H₄—NH—C₆H₄—OCH₃). The electroactive compound, NH₃⁺—C₆H₄—NH—C₆H₄—OCH₃, is detected amperometrically.

In other embodiments, measurements made by the SSMD may be free of any clotting reagent. For example, the impedance measurement used by the SSMD can yield the blood's viscosity, which is related to ACT by a pre-determined linear regression formula. This approach has an important advantage that it can be done very rapidly (e.g. in a matter of seconds), making quasi-continuous measurements of ACT a reality. In still other embodiments, a ‘hybrid’ measurement is made where the reagent-based ACT measurement is performed with a first electrode to yield an initial ‘ACT calibration’. The system then makes the above-described measurements of viscosity with a reagent-free electrode, and combines these with the ACT calibration to yield accurate (and rapid) follow-on measurements of ACT.

Clinical studies supporting the idea of using electrical impedance to measure the viscosity of a fluid (most notably human blood) are described in the following references, the contents of which are incorporated herein by reference: Pop et al., ‘On-line electrical impedance measurement for monitoring blood viscosity during on-pump heart surgery’, Eur Surg Res. 2004; 36(5):259-265 and Berney et al., ‘Impedance measurement monitors blood coagulation’, ADI. 2008; 42(3): 42-08. Relative changes in blood viscosity, in turn, indicate corresponding changes in Heparin levels and ACT, as described in the following references, the contents of which are incorporated herein by reference: Hitosugi et al., ‘Changes in blood viscosity by heparin and argatroban’, Thromb Res. 2001; 104(5):371-374. and Ranucci et al., ‘Blood viscosity during coagulation at different shear rates’, Physiol Rep. 2014; 2(7):e12065. Published 2014 Jul. 3. doi:10.14814/phy2.12065. This indicates that electrical impedance represents a potential measurement for determining relative changes in ACT in blood without using reagents.

Likewise, visible and near-infrared transmission optical spectroscopy is well established for determining H&H concentration from blood samples processed with clotting reagents as describe in the following references, the contents of which are incorporated herein by reference: Whitehead et al., ‘Methods and analyzers for hemoglobin measurement in clinical laboratories andfield settings’, Ann NY Acad Sci. 2019; 1450(1):147-171. Recent work indicates that relative changes in these parameters can yield results using comparable optical techniques and no reagents, as described in the following references, the contents of which are incorporated herein by reference: Zhang et al., ‘Nondestructive Measurement of Hemoglobin in Blood Bags Based on Multi-Pathlength VIS-NIR Spectroscopy’, Sci Rep. 2018; 8(1):2204. Published 2018 Feb. 2. doi:10.1038/s41598-018-20547-2.

The system above is described for use with human patients. In other embodiments, the system can be used with animals for veterinary applications. Such a system, for example, is typically a wearable system, as described above.

These and other embodiments of the invention are deemed to be within the scope of the following claims.

What is claimed is:

1. A system for automatically measuring a value of activated clotting time (ACT) from a blood sample from a patient, comprising:
a processing system;
a blood-extraction component controlled by the processing system and configured to extract the blood sample from the patient;
a measurement component connected to the blood-extraction component and controlled by the processing system, the measurement component configured to receive the blood sample from the blood-extraction component and deposit it in a measurement cell and comprising sensors configured to measure signals related to coagulation from the blood sample in the measurement cell; and
computer code running on the processing system that analyzes the signals to determine ACT from the blood sample.

2. The system of claim 1, wherein the blood-extraction component further comprises a syringe coupled to a motor-controlled actuator.

3. The system of claim 2, wherein the blood-extraction component further comprises a motorized valve.

4. The system of claim 3, wherein the processing component is configured to control the motor-controlled actuator to remove the blood sample from the patient.

5. The system of claim 4, wherein the processing component is configured to control the motor-controlled actuator and the motorized valve to pass blood to the measurement component after it is removed from the patient.

6. The system of claim 1, wherein the blood-extraction component further comprises a catheter.

7. The system of claim 6, wherein the blood-extraction component further comprises a manifold that connects to the catheter.

8. The system of claim 7, wherein the blood-extraction component further comprises a syringe coupled to both the manifold and a motor-controlled actuator.

9. The system of claim 8, wherein the catheter is configured to insert into an artery of the patient to extract the blood sample.

10. The system of claim 6, wherein the blood-extraction component further comprises a retractable sheath.

11. The system of claim 10, wherein the retractable sheath is configured to insert into the catheter.

12. The system of claim 11, wherein the retractable sheath is configured to extract blood once inserted into the catheter, and then be withdrawn from the catheter once blood is extracted.

13. The system of claim 12, wherein the catheter is configured to insert into a vein of the patient to extract the blood sample.

14. The system of claim 1, wherein the sensors comprised by the measurement component include at least one of the following for measuring the blood sample within the measurement cell to determine signals related to coagulation: 1) an optical sensor; 2) an electrical sensor for measuring impedance; 3) an electrical sensor for measuring reactance; 4) a camera for capturing images; and 5) a mechanical sensor for measuring propagation of an object within the measurement cell.

15. The system of claim 14, wherein the computer code running on the processing system is configured to operate an algorithm that analyzes the signals related to coagulation to calculate ACT.

16. The system of claim 14, wherein the sample cell encloses a compound for activating clotting within the blood sample once it is deposited in the sample cell.

17. The system of claim 16, wherein the compound is diatomaceous earth or a derivative thereof.

18. A system for automatically measuring a value of a parameter from a blood sample from a patient, comprising:
a processing system;
a blood-extraction component controlled by the processing system and configured to extract the blood sample from the patient;
a measurement component connected to the blood-extraction component and controlled by the processing system, the measurement component configured to receive the blood sample from the blood-extraction component and deposit it in a measurement cell and comprising sensors configured to measure signals related to the parameter from the blood sample in the measurement cell; and
computer code running on the processing system that analyzes the signals to determine a value of the parameter from the blood sample.

19. The system of claim 18, wherein the blood-extraction component further comprises a syringe coupled to a motor-controlled actuator.

20. The system of claim 19, wherein the blood-extraction component further comprises a motorized valve.

21. The system of claim 20, wherein the processing component is configured to control the motor-controlled actuator to remove the blood sample from the patient.

22. The system of claim 21, wherein the processing component is configured to control the motor-controlled actuator and the motorized valve to pass blood to the measurement component after it is removed from the patient.

23. The system of claim 18, wherein the blood-extraction component further comprises a catheter.

24. The system of claim 23, wherein the wherein the blood-extraction component further comprises a manifold that connects to the catheter.

25. The system of claim 24, wherein the blood-extraction component further comprises a syringe coupled to both the manifold and a motor-controlled actuator.

26. The system of claim 25, wherein the catheter is configured to insert into an artery of the patient to extract the blood sample.

27. The system of claim 23, wherein the blood-extraction component further comprises a retractable sheath.

28. The system of claim 27, wherein the retractable sheath is configured to insert into the catheter.

29. The system of claim 28, wherein the retractable sheath is configured to extract blood once inserted into the catheter, and then be withdrawn from the catheter once blood is extracted.

30. The system of claim 29, wherein the catheter is configured to insert into a vein of the patient to extract the blood sample.

31. The system of claim 18, wherein the sensors comprised by the measurement component include at least one of the following for measuring the blood sample within the measurement cell to determine signals related to coagulation: 1) an optical sensor; 2) an electrical sensor for measuring impedance; 3) an electrical sensor for measuring reactance; 4) a camera for capturing images; and 5) a mechanical sensor for measuring propagation of an object within the measurement cell.

32. The system of claim 31, wherein the computer code running on the processing system is configured to operate an algorithm that analyzes the signals to calculate a value of the parameter.

33. The system of claim 31, wherein the sample cell encloses a compound configured to chemically react with the blood sample once it is deposited in the sample cell.

34. The system of claim 33, wherein the compound is a chemical reagent.

35. A system for automatically measuring a parameter related to coagulation from a blood sample, the system comprising a computer-controlled blood-extraction component that extracts the blood sample from a patient and a computer-controlled measurement component that receives the blood sample from the blood-extraction component and with a sensor measures signals from the blood sample that relate to coagulation to determine a value of the parameter.

36. The system of claim 35, configured to be worn entirely on the patient's body.
